SEQUENCE LISTING (4 sequences)

SEQ ID NO: 1 - DNA, 21 bp, Artificial sequence
  Antisense oligonucleotide
gtaatcatcttgattacccca

SEQ ID NO: 2 - protein, 480 aa, Homo sapiens
MAYSQGGGKKKVCYYYDGDIGNYYYGQGHPMKPHRIRMTHNLLLNYGLYRKMEIYRPHKATAEEMTKYHSDEYIKFLRSIRPDNMSEYSKQMQRFNVGEDCPVFDGLFEFCQLSTGGSVAGAVKLNRQQTDMAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNFPMRDGIDDESYGQIFKPIISKVMEMYQPSAVVLQCGADSLSGDRLGCFNLTVKGHAKCVEVVKTFNLPLLMLGGGGYTIRNVARCWTYETAVALDCEIPNELPYNDYFEYFGPDFKLHISPSNMTNQNTPEYMEKIKQRLFENLRMLPHAPGVQMQAIPEDAVHEDSGDEDGEDPDKRISIRASDKRIACDEEFSDSEDEGEGGRRNVADHKKGAKKARIEEDKKETEDKKTDVKEEDKSKDNSGEKTDTKGT

SEQ ID NO: 3 - protein, 367 aa, Homo sapiens
MVMEVGTLDAGGLRALLGERAAQCLLLDCRSFFAFNAGHIAGSVNVRFSTIVRRRAKGAMGLEHIVPNAELRGRLLAGAYHAVVLLDERSAALDGAKRDGTLALAAGALCREARAAQVFFLKGGYEAFSASCPELCSKQSTPMGLSLPLSTSVPDSAESGCSSCSTPLYDQGGPVEILPFLYLGSAYHASRKDMLDALGITALINVSANCPNHFEGHYQYKSIPVEDNHKADISSWFNEAIDFIDSIKNAGGRVFVHCQAGISRSATICLAYLMRTNRVKLDEAFEFVKQRRSIISPNFSFMGQLLQFESQVLAPHCSAEAGSPAMAVLDRGTSTTTVFNFPVSIPVHSTNSALSYLQSPITTSPSC

SEQ ID NO: 4 - protein, 589 aa, Homo sapiens
MDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

(19) United States
(12) Patent Application Publication
(10) Pub. No.: US 2012/0022129 A1
(43) Pub. Date: Jan. 26, 2012

(54) Targeting of Histone Deacetylase 2, Protein Kinase CK2, and Nuclear Factor NRF2 For Treatment of Inflammatory Diseases

(76) Inventor: Irfan Rahman, Rochester, NY (US)

(21) Appl. No.: 13/030,354

(22) Filed: Feb. 18, 2011

Related U.S. Application Data
(63) Continuation-in-part of application No. PCT/US2009/054152, filed on Aug. 18, 2009.
(60) Provisional application No. 61/089,752, filed on Aug. 18, 2008.
(60) Provisional application No. 61/415,591, filed on Nov. 19, 2010.

(51) Int. Cl. A61K 31/7088; A61K 31/4192; A61K 31/56; A61K 31/352; A61K 31/26; A61K 31/497; A61K 31/277; A61K 31/05; A61K 31/385; A61K 31/12; A61K 31/216; A61P 11/00; A61P 11/06; A61P 29/00; A61P 1/00; A61K 31/711

(52) U.S. Cl. 514/44 A; 514/359; 514/171; 514/456; 514/514; 514/255.05; 514/519; 514/731; 514/733; 514/441; 514/679; 514/532

(57) ABSTRACT

Methods for the treatment or prevention of diseases which are caused by the degradation of histone deacetylase 2 (HDAC2) in cells are described. The diseases which may be treated by the methods of the invention include chronic obstructive pulmonary disease (COPD) and asthma. The invention provides methods for treating or preventing of diseases caused by the degradation of HDAC2 by providing to the subject in need of treatment or prevention a molecular compound capable of preventing the degradation of HDAC2. Such molecular compounds include protein kinase CK2 inhibitors, ubiquitination inhibitors, ubiquitin-proteosome inhibitors, nuclear factor (erythroid-derived 2)-like 2 (Nrf2) activators and MAPK phosphatase 1 activators. Methods are also provided for the treatment and prevention of diseases caused by the degredation of HDAC2 by interfering with the expression of protein kinase CK2 or by increasing expression of Nrf2.

### CROSS REFERENCE TO RELATED APPLICATIONS

This application is a continuation-in-part of International Patent Application No. PCT/US2009/054152, filed Aug. 18, 2009, which claims priority to U.S. Provisional Patent Application No. 61/089,752, filed Aug. 18, 2008, which are hereby incorporated by reference herein. This application also claims priority to U.S. Provisional Patent Application No. 61/415,591, filed Nov. 19, 2010, which is hereby incorporated by reference herein.

### STATEMENT REGARDING FEDERALLY-SPONSORED RESEARCH AND DEVELOPMENT

The subject matter of this application was made with support from the United States Government under Grant No. R01-H1-085613 from the National Institutes of Health. The United States Government has certain rights in the invention.

### BACKGROUND OF THE INVENTION

1. Field of the Invention

The present invention relates to methods of treating inflammatory diseases by modulating levels and/or activity of histone deacetylase 2 (HDAC2) through either direct modulation or modulation of factors which affect HDAC2 levels and/or activity as well as overcoming the steroid resistance.

2. Background Art

Chronic obstructive pulmonary diseases (COPD) are conditions of the lungs, including emphysema and chronic bronchitis, in which the airways of the lungs are narrowed, making it difficult to breath. While exposures to other air pollutants can cause COPD, the primary cause of the disease is exposure to cigarette smoke. The inflammation that leads to COPD has been associated with the expression of proinflammatory genes driven by the Nuclear Factor KB (NF-κB) transcription factor complex (14). There is no cure for COPD, but subjects are often treated with corticosteroids to help slow the progression of the disease. The long term treatment of COPD with corticosteroids often leads to steroid resistance, whereby the treatment becomes progressively less effective.

Cigarette smoke (CS) is a complex mixture of oxidants/free radicals and different chemical compounds which include reactive aldehydes and quinones known to cause oxidative stress in the lungs (1), is the primary risk factor for the pathogenesis of COPD(2, 3). It is believed that cigarette smoking is the primary etiological factor in steroid resistance observed in subjects with COPD and asthmatics who smoke (4), a phenomenon which is also observed in other inflammatory diseases, such as rheumatoid arthritis (5) and inflammatory bowel disease (6). On ligand binding, corticosteroids suppress inflammation by glucocorticoid receptor recruitment of histone deacetylase 2 (HDAC2) specifically to acetylated histones on promoters of proinflammatory genes such as IL-8 and GM-CSF (7).

Histone deacetylases (HDACs) are a family of cellular enzymes that regulate gene expression by catalyzing the removal of acetyl groups from the lysine tails of core histones (8). HDAC2 is a class I histone deacetylase that resides almost exclusively in the nucleus and is a critical part of co-repressor complexes recruited to proinflammatory gene promoters by associated proteins (8). The inability of corticosteroids to recruit HDAC2 or the presence of post-translationally modified HDAC2 may explain the abnormal inflammatory response and ineffectiveness of corticosteroid therapy in subjects with COPD (9-11). In animal exposure experiments, lungs of rats and different strains of mice exposed to cigarette smoke (CS) exhibit significantly decreased HDAC2 expression and activity similar to that observed in peripheral blood mononuclear cells (PBMCs) of subjects with mild to severe asthma, alveolar macrophages of COPD subjects and chronic asthmatics who smoke (9-13). Bronchial biopsies of COPD subjects and asthmatics who smoke exhibit decreased HDAC2 expression which correlates with disease severity, increased cytokine production and corticosteroid insensitivity (14, 15). Several studies have suggested that corticosteroid insensitivity is closely related to reduced expression of HDAC2, Increased post-translational modification and subsequently degradation of HDAC2 required to balance increased IKKα-mediated chromatin modification (acetylation of histone proteins) of proinflammatory promoters in lungs in response to CS (16, 17). In view of this, it has been recently shown that HDAC2 is modified by nitration of tyrosine residues, formation of protein-aldehyde adducts in response to CS, or by reactive aldehydes subsequently leading to loss of HDAC2 activity (10, 11). Despite overwhelming evidence that loss of HDAC2 by oxidant-mediated post-translational modification is closely linked to corticosteroid insensitivity, the molecular mechanisms of CS-induced degradation of HDAC2, particularly relating to kinase signaling in vivo and in vitro, are still unclear.

Recently, it has been shown that phosphorylation may play a more important role in regulating HDAC2 trans-repression ability, enzymatic activity and expression (18, 19), Furthermore, basal HDAC2 expression in cells is mediated via the ubiquitin-proteasome pathway (20), but no studies have been conducted to determine whether a phosphorylation-ubiquitination-proteasome pathway is involved in the cellular loss of HDAC2 in response to CS, oxidants or any environmental stimuli.

As steroid resistance in COPD can lead to the loss of ability to control the symptoms of the disease. Other compounds, such as theophylline, have been used as add-on therapies to counter steroid resistance (36). However, due to the numerous side-effects of theophylline, its use has been largely discontinued. Therefore, there remains a need in the art for treatments of COPD inflammation that are effective and safe either alone or in combination with corticosteroid therapies.

### BRIEF SUMMARY OF THE INVENTION

It is an object of the present invention to provide methods for the treatment and prevention of diseases related to reduced cellular levels of histone deacetylase 2 (HDAC2) in a subject. The diseases which may be treated or prevented by the methods of the present invention are caused by reduced cellular levels of HDAC2, which can lead to the expression of genes involved in inflammation which are driven by the NF-κB promoter. The diseases which may be treated by the methods of the present invention include chronic obstructive pulmonary disease (COPD) and asthma, among others.

It is as object of the present invention to provide methods for the treatment and prevention of diseases related to reduced cellular levels of HDAC2 in a subject by providing to the subject a molecular compound which is capable of maintaining cellular levels of HDAC2. Such molecular compounds include protein kinase CK2 (CK2) inhibitors, ubiquitination inhibitors, ubiquitin-proteosome inhibitors, and MAPK phosphatase 1 (MKP1) activators. Other molecular compounds include Nuclear factor (erythroid-derived 2)-like 2 (Nrf2) activators.

It is a further object of the present invention to provide methods for the treatment and prevention of diseases related to reduced cellular levels of HDAC2 in a subject by providing to the subject an oligonucleotide capable of interfering with the expression of CK2. The oligonucleotide may be delivered using methods know in the art and may be delivered directly to lung cells in order to effect this result.

It is an object of the present invention to provide methods for the treatment and prevention of COPD and asthma in a subject by providing to the subject a nucleic acid sequence which causes expression of HDAC2, Nrf2a, or both HDAC2 and Nrf2 in the lung cells of the subject.

It is a further object of the present invention to provide methods for the treatment and prevention of diseases related to reduced cellular levels of HDAC2 in a subject by providing to the subject a molecular compound which is capable of maintaining cellular levels of HDAC2. Such molecular compounds include Nrf2 activators.

It is a further object of the present invention to provide methods for the treatment and prevention of COPD and asthma in a subject by providing to the subject a nucleic acid sequence which causes expression of HDAC2 or a similar protein in the lung cells of the subject.

It is a still further object of the present invention to provide methods for the treatment and prevention of diseases related to reduced cellular levels of HDAC2 in a subject by providing to the subject a nucleic acid sequence which causes the expression of MKP1 or similar protein in the lung cells of the subject.

### DETAILED DESCRIPTION OF THE DRAWINGS/FIGURES

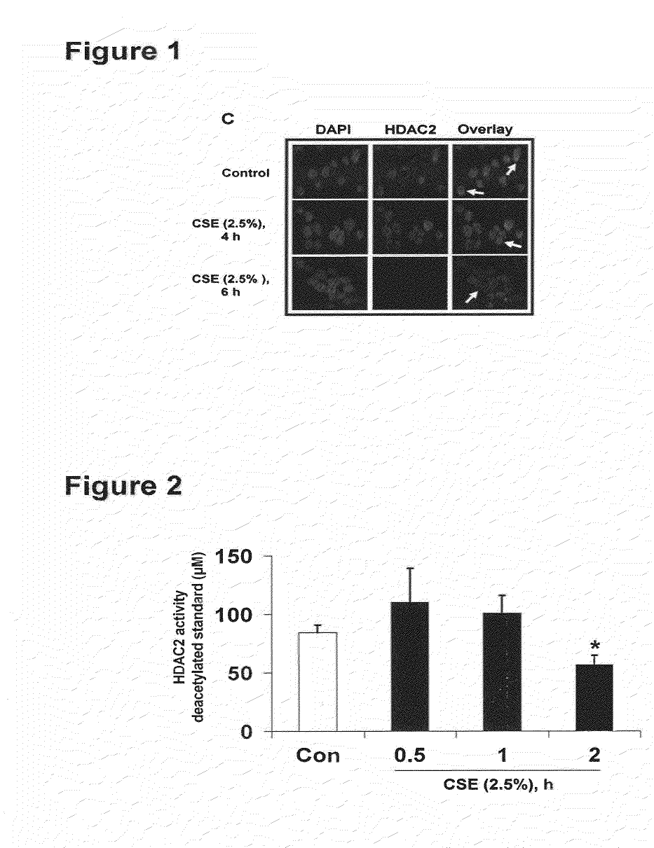

FIG. 1: Cigarette smoke extract (CSE) time-dependently caused degradation of class I HDACs in MonoMac6 cells. MonoMac6 cells were treated with CSE (2.5%) for indicated time points 10 μg of protein from whole cell extracts was electrophoresed on SDS 7.5% polyacrylamide gel, transferred onto PVDF membranes and immunochemically stained with HDAC1, HDAC2 and HDAC3 antibodies. (A) CSE significantly decreased class I HDAC levels as early as 1 h after CSE exposure. (B) Relative density of HDAC2 levels in human macrophage cells (MonoMac6 cells) at various time points of exposure to CSE. (C) MonoMac6 cells were treated with CSE (2.5%) for 0, 4 and 6 h, cytospin slides were prepared and cells fixed in 4% paraformaldehyde and immunostained with anti-HDAC2 antibodies. DAPI stain was used to visualize the nucleus (shown by arrows), CSE showed significant loss of HDAC2 expression in the nucleus without any nucleo-cytoplasmic shuttling in MonoMac6 cells. Data are shown as mean±SEM (n=3)*** P<0.001, significant compared with control treatments.

FIG. 2: CSE induced HDAC2 degradation is associated with decreased specific HDAC2 activity in MonoMac6 cells. MonoMac6 cells were treated with CSE (2.5%) for 0.5, 1 and 2 h, immunoprecipitated HDAC2 (500 μg protein) from whole cell extracts was assayed for HDAC2 activity with a colorimetric assay kit (Biomol, Int., Plymouth Meeting, Pa.) at 405 nm with COLOR DE LYS substrate (500 μM) and expressed as deacetylated standard using a 1 μM-500 μM standard curve. HeLa nuclear extract was used as a positive control data are shown as mean±SEM (n=4) * P<: 0.05, significant compared to media alone control treatments.

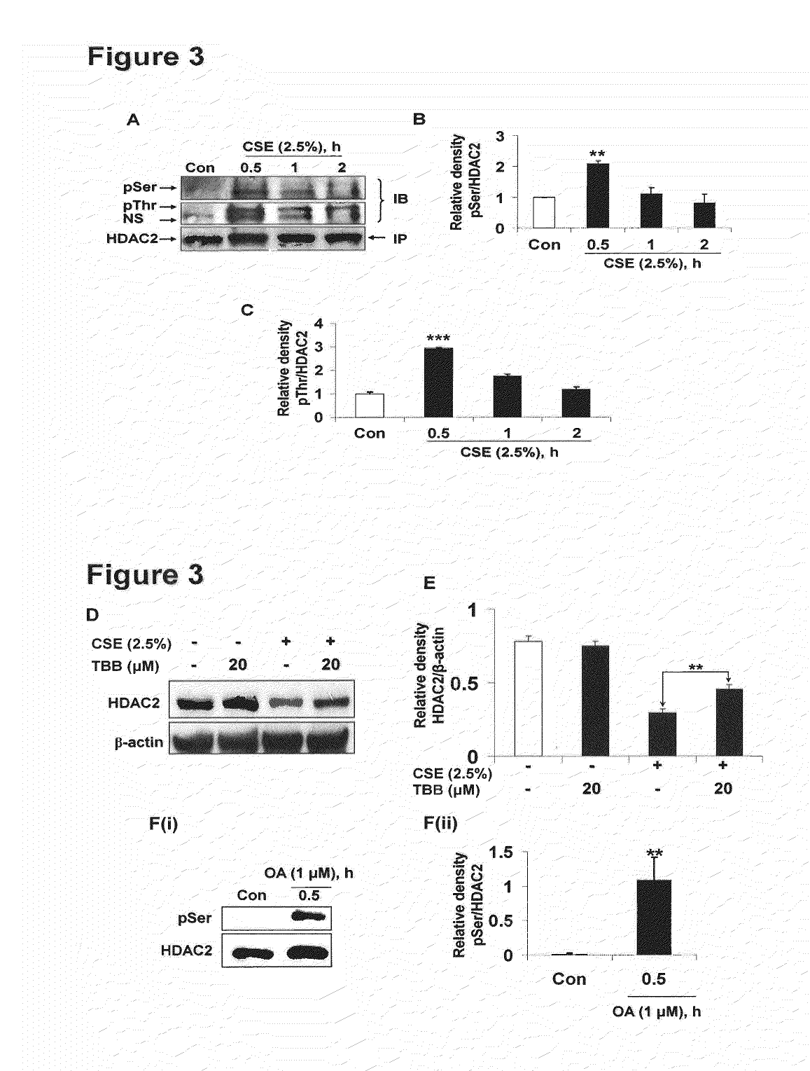

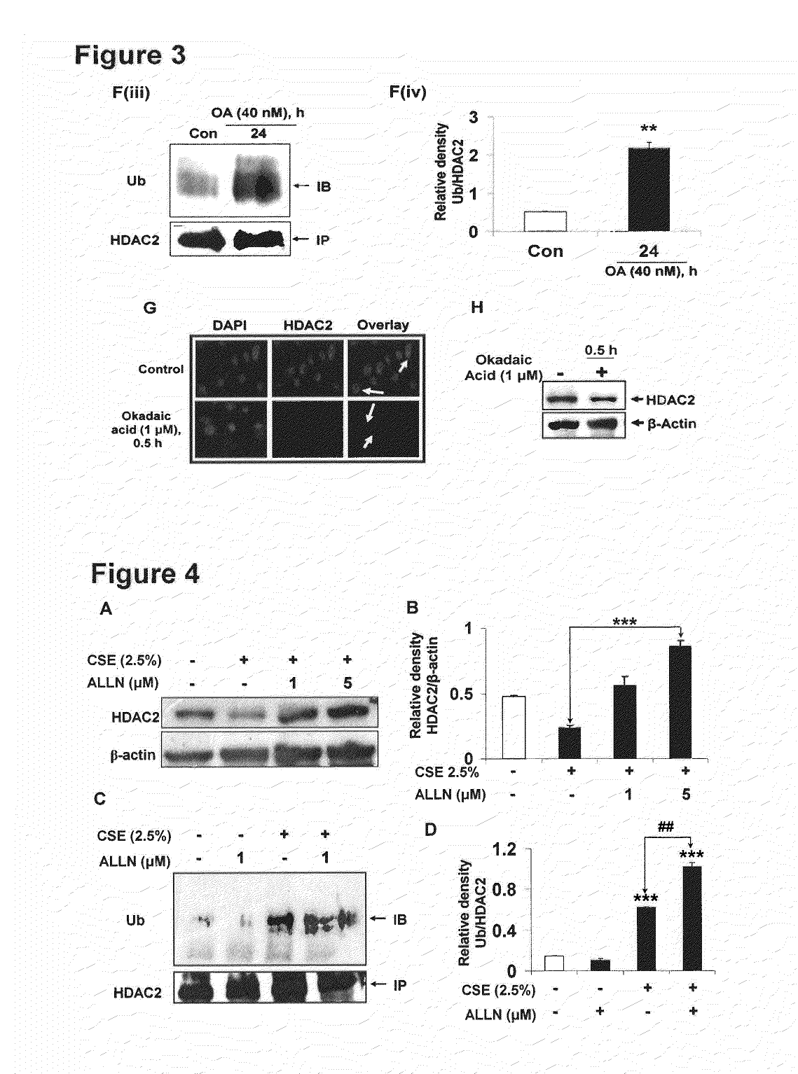

FIG. 3: CSE induces HDAC2 phosphorylation via protein casein kinase CK2 in MonoMac6 cells. (A) MonoMac6 cells were treated with CSE (2.5%) for 0.5, 1 and 2 h immunoprecipitated HDAC2 was analyzed for phosphorylation of serine and threonine residues using specific phospho-serine and phospho-threonine antibody by SDS-polyacrylamide gel. CSE induced significant phosphorylation of HDAC2 on serine and threonine residues. Relative density of (B) phospho-serine and (C) phospho-threonine expression normalized to immunoprecipitated HDAC2. (D) MonoMac6 cells were pre-treated with or without protein kinase CK2 inhibitor [20 μM 4,5,6,7-tetrabromobenzotriazole (TBB)] for 2 h, Cells were then exposed to CSE (2.5%) for 6 h. Ten μg protein from whole cell lysates was electrophoresed on SDS-polyacrylamide gel and assayed for HDAC2 levels. TBB significantly inhibited HDAC2 degradation in response to CSE. (E) Relative density of HDAC2 levels normalized to β-actin. [F (i)] MonoMac6 cells were treated with phosphatase inhibitor, okadaic acid, (1 μM) for 0.5 h. Immunoprecipitated HDAC2 was analyzed for phosphorylation of serine residues using a specific phospho-serine antibody by SDS-polyacrylamide gel. Okadaic acid significantly induced phosphorylation of HDAC2 on serine residues, [F (ii)] Relative density of phospho-serine expression normalized to immunoprecipitated HDAC2. [F (iii)] MonoMac6 cells were treated with or without phosphatase inhibitor, okadaic acid, (40 nM) for 24 h. Immunoprecipitated HDAC2 was assessed for bound ubiquitin using specific ubiquitin anti-sera. Okadaic acid induced significant ubiquitination of HDAC2, [F (iv)] Relative density of ubiquitin levels normalized to immunoprecipitated HDAC2, (G) MonoMac6 cells were treated with okadaic acid (1 μM) for 0.5 h, cytospin slides were prepared and cells fixed in 4% paraformaldehyde and immunostained with anti-HDAC2 antibodies DAPI stain was used to visualize the cell nucleus, Okadaic acid showed an early and significant loss of HDAC2 expression in the nucleus without any nucleo-cytoplasmic shuttling in MonoMac6 cells. Data are expressed as mean±SEM (n=3) * P<0.05, *** P<0.01, significant compared to media alone treatments.

FIG. 4: CSE-mediated degradation of HDAC2 is ubiquitin-proteasome dependent in MonoMac6 cells. (A) MonoMac6 cells were pre-treated with proteasome inhibitor N-Acetyl-LeuLeu-NIe-CHO (ALLN, 1 μM and 5 μM) for 2 h followed by a 6 h exposure to CSE (2.5%). Ten μg of protein from whole cell extracts was electrophoresed on an SDS 7.5% polyacrylamide gel, transferred onto PVDF membranes and immunochemically stained with HDAC2 antibodies. Dose-dependent inhibition of the proteasome significantly attenuated HDAC2 degradation. (B) The relative density of HDAC2 expression in MonoMac6 cells after proteasome inhibition of CSE induced HDAC2 degradation normalized to β-actin. (C) MonoMac6 cells were pre-treated with or without ALLN (1 μM) and then exposed to CSE 2.5% for 6 h. Immunoprecipitated HDAC2 was assayed for bound ubiquitin using specific ubiquitin anti-sera. CSE induced significant increase in HDAC2 ubiquitination which was enhanced with ALLN pre-treatment. (D) The relative density of ubiquitin expression in MonoMac6 cells after proteasome inhibition of CSE-induced HDAC2 degradation normalized to immunoprecipitated HDAC2. Data are shown as mean±SEM (n=3) *** P<0.001, significant compared to media alone control treatments.

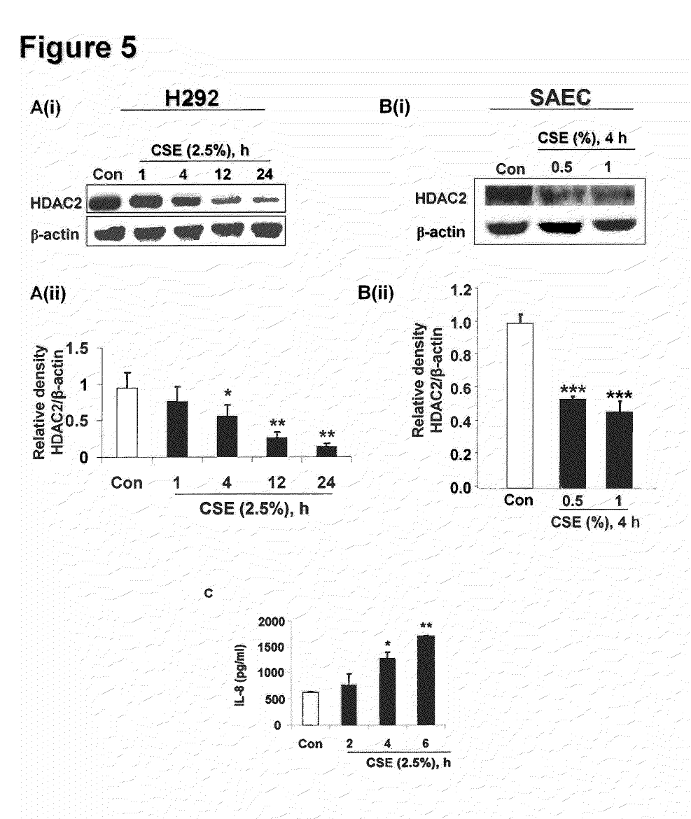

FIG. 5: CSE time-dependently caused degradation of HDAC2 associated with increased cytokine release in human bronchial epithelial and human primary small airway epithelial cells (SAEC). [A (i)] Human bronchial epithelial cells (H292) were treated with CSE (2.5%) for 0, 1, 4, 12, 24 h. Ten μg of protein from whole cell extracts was electrophoresed on an SDS 7.5% polyacrylamide gel, transferred onto PVDF membranes and immunochemically stained with HDAC2 antibodies, CSE time-dependently decreased HDAC2 expression in H292 cells. [A (ii)] The relative density of HDAC2 expression in H292 cells exposed to CSE normalized to β-actin. [B (i)] Small airway epithelial cells (SAEC) were exposed 10 CSE (0.5% and 1%) for 4 h. Whole cell lysates were electrophoresed on SDS polyacrylamide gels and membranes immunochemically stained for HDAC2 expression, CSE time-dependently induced HDAC2 degradation. [B (ii)] Relative density of HDAC2 expression in SAECs exposed to CSE normalized to I)-actin. (C) Conditioned media from H292 cells treated with CSE (2.5%) were assayed for IL-8 levels by sandwich ELISA. CSE induced an increase in IL-8 release with LPS used as a positive control. Data are shown as mean±SEM (n=3). * P<0.05, ** P<0.01, ***. P<0.001, significant compared to control treatments.

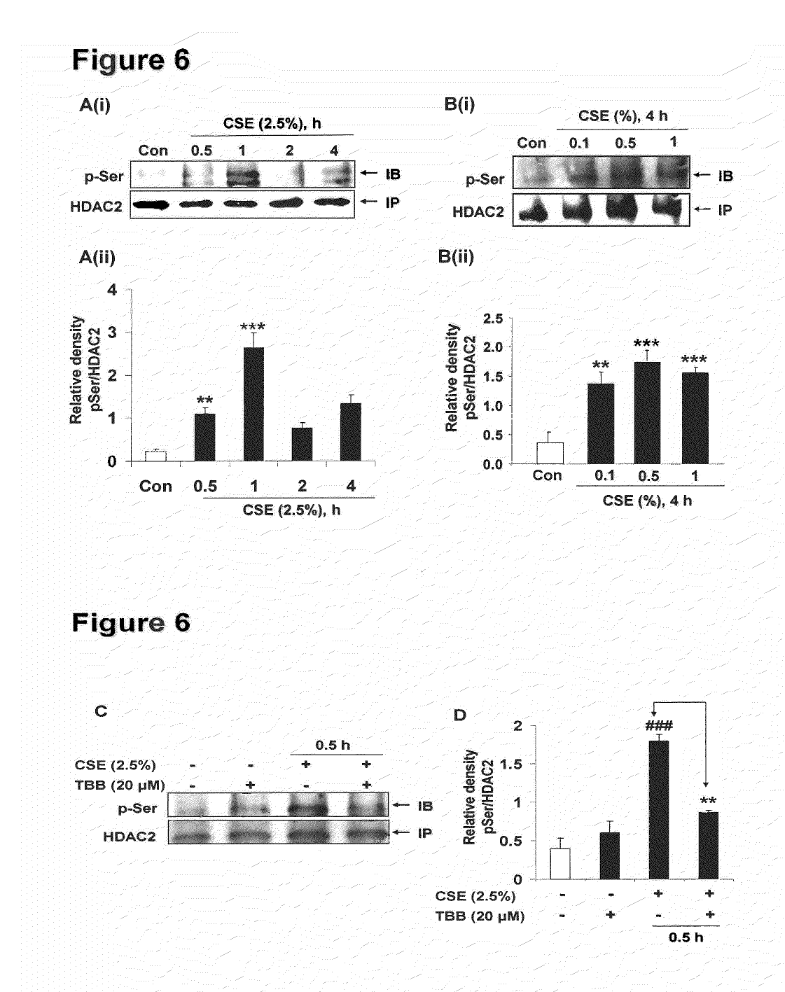

FIG. 6: CSE induced CK2-dependent HDAC2 phosphorylation in bronchial epithelial and small airway epithelial cells. [A (i)] H292 cells were treated with CSE (2.5%) for 0.5, 1, 2 and 4 h. Immunoprecipitated HDAC2 was analyzed for phosphorylation of serine residues using specific phospho-serine antibodies by SDS polyacrylamide gel. CSE induced significant cyclical phosphorylation of HDAC2 on serine residues at 0.5, 1 and 4 h. [A (ii)] Relative density of phosphor-serine expression normalized to immunoprecipitated HDAC2. [B (i)] SAECs were exposed to CSE (0.1%, 0.5% and 1%) for 4 h. Immunoprecipitated HDAC2 from whole cell lysates was probed for phosphorylation of serine residues, CSE induced a dose-dependent increase in HDAC2 serine phosphorylation. [B (ii)] Relative density of phospho-serine expression normalized to immunoprecipitated HDAC2. (C) H292 cells were treated with or without specific CK2 inhibitor, TBB. for 1 h, cells were washed with sterile 1×PBS and then treated with freshly prepared CSE (2.5%) for 0.5 h, Immunoprecipitated HDAC2 was assayed for phosphorylation on serine residues. TBB significantly inhibited CSE-induced HDAC2 phosphorylation. (D) Relative density of phosphor-serine expression normalized to immunoprecipitated HDAC2. Data are expressed as mean±SEM (n=3). *P<0.05, ** P<0.01, *** P<0.001, ### P<: 0.001 significant compared to control treatments.

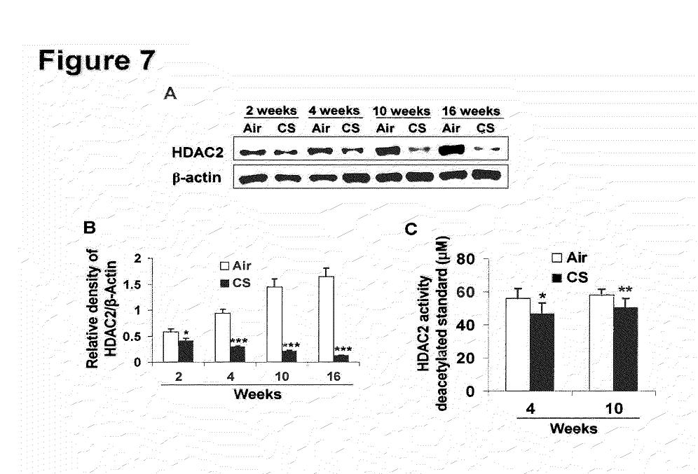

FIG. 7: Chronic CS exposure caused HDAC2 degradation and decreased activity in mouse lungs. Mice were exposed to CS as described in the Materials and Methods of Example 1. Lung tissue (100-150 mg) was homogenized and nuclear extracts prepared and frozen until analyzed. Five μg protein of nuclear extracts were analyzed for HDAC2 levels on SDS-polyacrylamide gels. (A) CS time-dependently decreased HDAC2 expression in mouse lungs. (B) Relative density of HDAC2 expression normalized to β-actin. (C) HDAC2 was immunoprecipitated and HDAC2 activity measured by colorimetric assay kit (Biomol, Int., Plymouth Meeting, Pa.) using COLOR DE LYS substrate. Mice exposed to CS showed decreased specific HDAC2 activity as compared to air exposed mice. Data are expressed as mean±SEM (n=4-5 mice per group), * P<0.05, ** P<0.01, *** P<0.001, significant compared to air exposed mice.

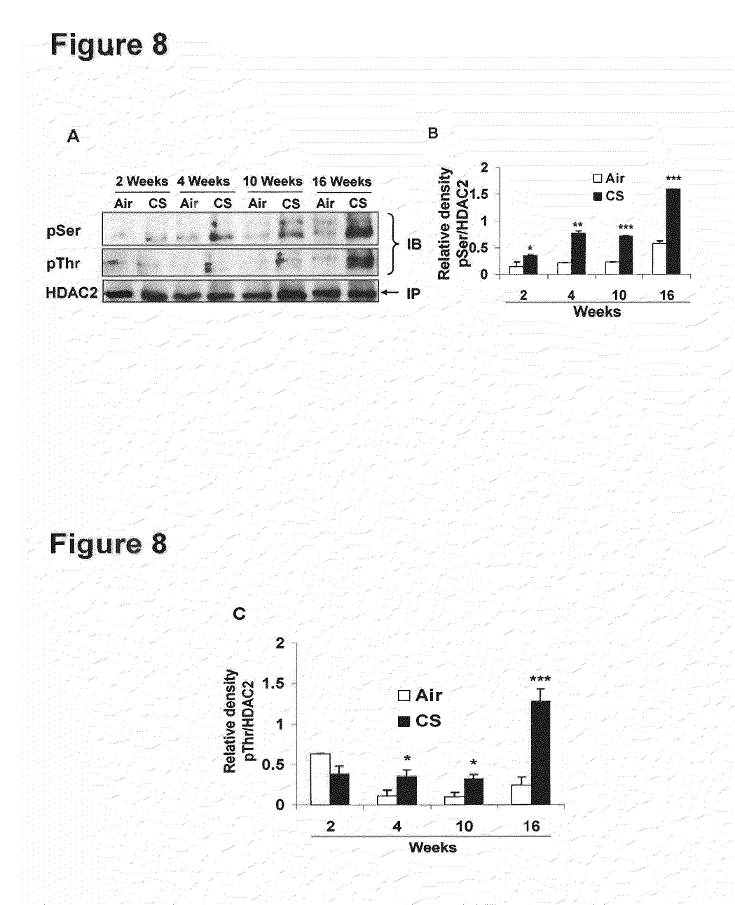

FIG. 8: CS induces HDAC2 phosphorylation in mouse lungs. Mice were exposed to CS as described in the Materials and Methods of Example 1. Mouse lung tissue (100-150 mg) was homogenized in RIPA buffer (100 μl) as whole tissue extracts and HDAC2 (500 μg) was immunoprecipitated. Phosphorylation of serine and threonine residues was analyzed using specific phospho-serine and phospho-threonine antibodies on an SDS polyacrylamide gel. (A) Mice exposed to CS for 2, 4, 10 and 16 weeks showed significant phosphorylation of HDAC2 on both serine residues, however, mice exposed at 2 weeks did not show significant phosphorylation on threonine residues, Relative density of (B) phospho-serine and (C) phospho-threonine HDAC2 expression normalized to immunoprecipitated HDAC2. Data are expressed as mean±SEM (n=4-5 mice per group) * P<0.05, ** P<0.01, *** P<0.001, significant compared to air exposed littermates.

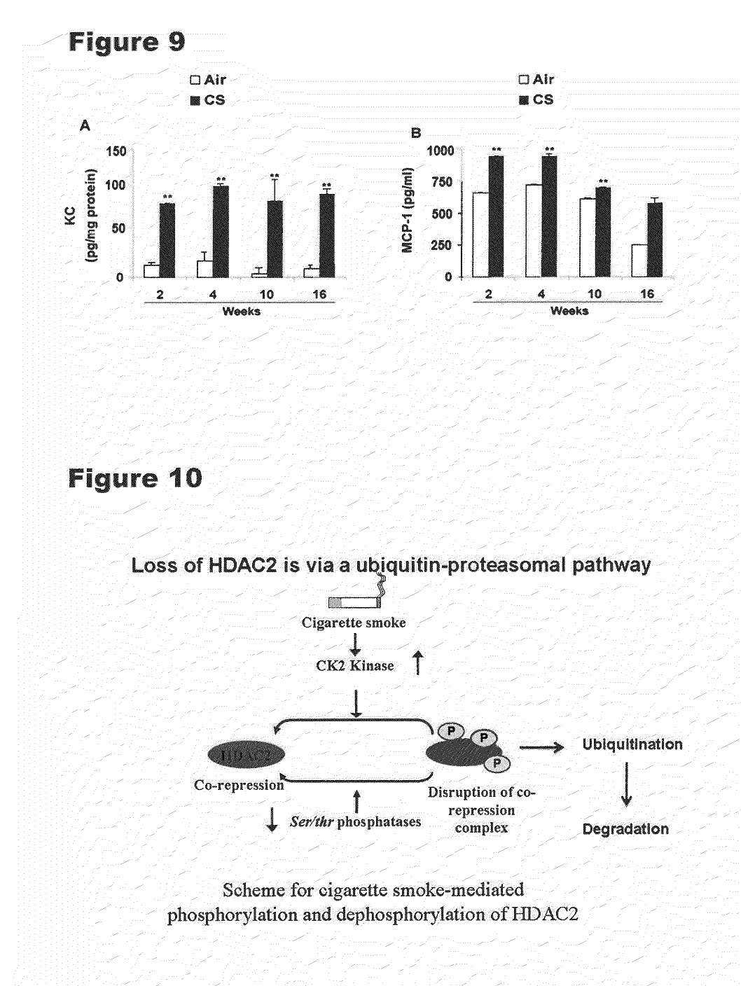

FIG. 9: CS induces increased cytokine release in mouse lung tissue. Soluble fractions of mouse lung tissue were analyzed for (D) KC and (E) MCP-1 by sandwich ELISA; values were normalized to total lung protein content. Data are expressed as mean±SEM (n=4-5 mice per group). * P<0.05, ** P<0.01 significant compared to air exposed littermates.

FIG. 10: A schematic of the degradation of HDAC2 through a ubiquitin proteosomal pathway.

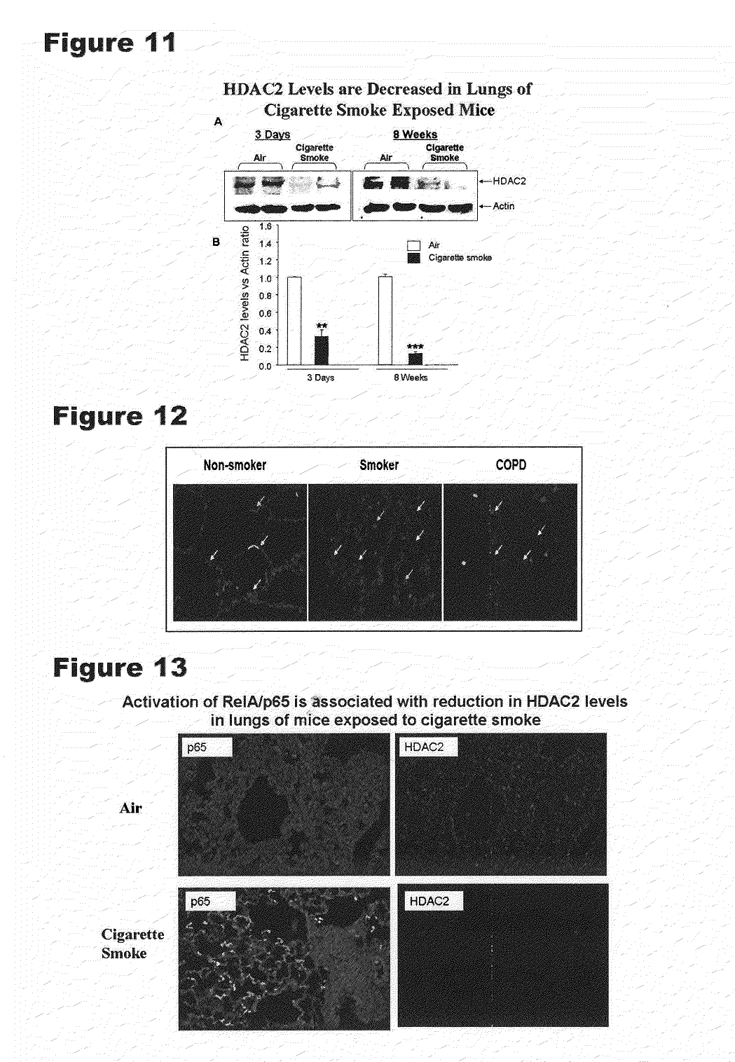

FIG. 11: Cigarette smoke exposure decreases HDAC2 levels in mouse lungs. Cigarette smoke exposure led to decreased levels of HDAC2 proteins at 24 hours (sacrifice time) post-last exposure after 3 days and 8 weeks exposures compared to air exposed group, after 3 days and 8 weeks exposures. (A) Western blots picture of soluble nuclear proteins extracted from lung homogenate. (B) Graph showing the relative density (ratio of control) of HDAC2. Gel picture shown is representative of three separate experiments (n=4 in each group). Air=Air-exposed mice. **p<0.01 and ***p<0.001, compared to air groups.

FIG. 12: Immunohistochemistry of human lung sections obtained from non-smokers, smokers with and without COPD showing decreased expression of HDAC2 in alveolar epithelial cells and macrophages. White arrows indicate type II alveolar epithelial cells and green arrows indicate macrophages. The picture is representative of three separate experiments (n=6 in each group).

FIG. 13: Immunohistochemistry of mouse lung sections air and CS groups showing decreased expression of HDAC2 in alveolar epithelial cells and macrophages associated NF-kB (RelA/p65) activation. The picture is representative of three separate experiments (n=6 in each group).

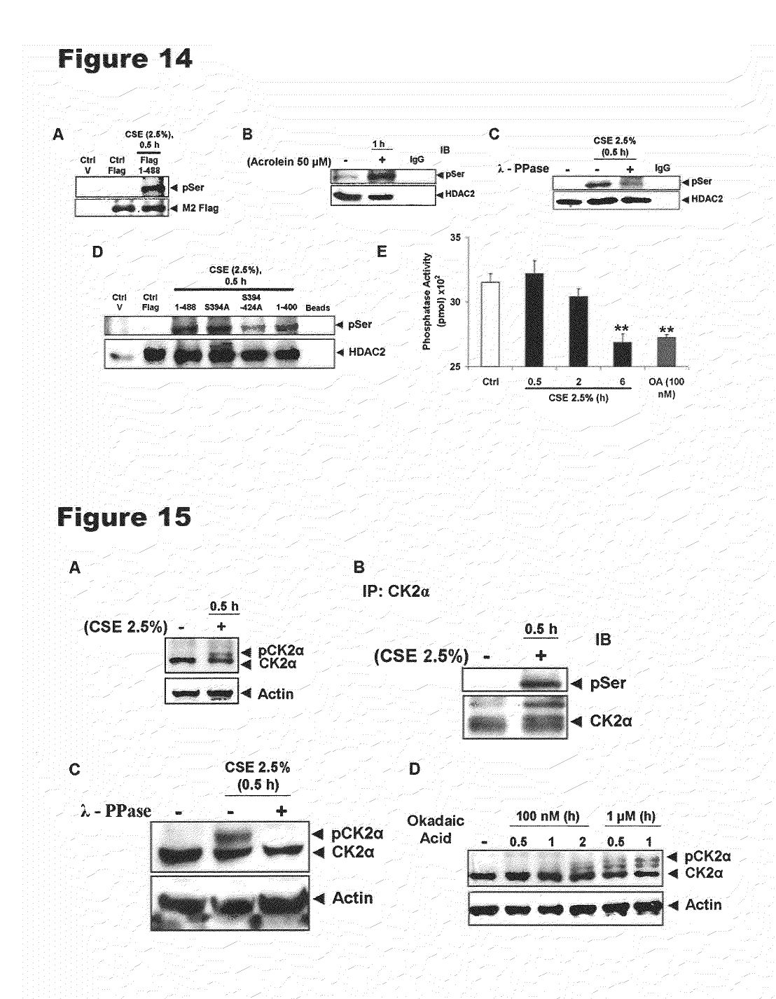

FIG. 14: Loss of Serine Sites S³⁹⁴, S⁴²², S⁴²⁴ Abrogates Cigarette Smoke Extract (CSE)-Induced Phosphatase-Independent HDAC2 Phosphorylation. A) Bronchial epithelial H292 cells were transfected with plasmid vector or flag-tagged WT HDAC2 (1-488) for 24 h using lipofectamine 2000 and then treated with or without 2.5% CSE for 0.5 h or B) 50 μM. acrolein for 1 h. Flag-tagged proteins were immunoprecipitated overnight and serine phosphorylation detected by western blots using phospho-serine antibodies. C) Flag-tagged WT HDAC2 was immunoprecipitated from whole cell lysates of H292 cells treated with 2.5% CSE for 0.5 h. Immunoprecipates were then washed and treated with 400 U of λ-phosphatase or buffer alone for 1 h at 30° C. Phosphorylation of serine sites were detected by western blots using anti-serine antibodies. D) H292 cells were transfected with plasmid vector, flag-tagged WT HDAC2 (1-488) or S394A, S394/411/422/424A and 1-400 aa HDAC2 mutants for 24 h with lipofectamine 2000. Cells were then treated with 2.5% CSE for 0.5 h. Serine phosphorylation of immunoprecipitated flag proteins was determined by western blot. E) Phosphatase activity was determined using non-radioactive assay system (Promega). H292 cells were treated with or without 2.5% CSE for 0.5, 2 and 6 h with serine/threonine phosphatase (okadaic acid) used as a positive control. 5 μg protein was incubated with PP2A specific-peptide substrate RRA(pT)VA and reaction buffer for 15 minutes in a 96-well plate. Molybdate dye/additive mixture was added to stop the reaction and color allowed to develop for 15 minutes. Optical density of samples was determined using a microplate reader with a 630 nm filter. Data shown as mean±SEM, ** p<0.01 significantly different from control groups.

FIG. 15: CK2α Is Phosphorylated In Response to CSE. A) 20 μg of whole cell lysates from bronchial epithelial H292 cells treated with or without 2.5% CSE for 0.5 h, were resolved on a 7.5% SDS/polyacrylamide gel and relative CK2α expression was determined by western blotting. B) CK2α immunoprecipitates from 500 μg of H292 cell lysates were resolved on a 7.5% SDS/polyacrylamide gel and serine phosphorylation was determined by western blotting using specific phospho-serine antibodies, C) H292 cells were treated with or without 2.5% CSE for 0.5 h. 80 μg whole cell lysates were then treated with 400 U of λ-phosphatase or buffer alone for 1 h at 30° C. The reaction was stopped by adding equal volume of 2× sample buffer and relative CK2α expression determined by western blotting. D) H292 cells were treated with 100 nM and 1 μM okadaic acid for 0.5 and 1 h. Relative CK2α expression was determined by western blotting using 20 μg whole cell lysates.

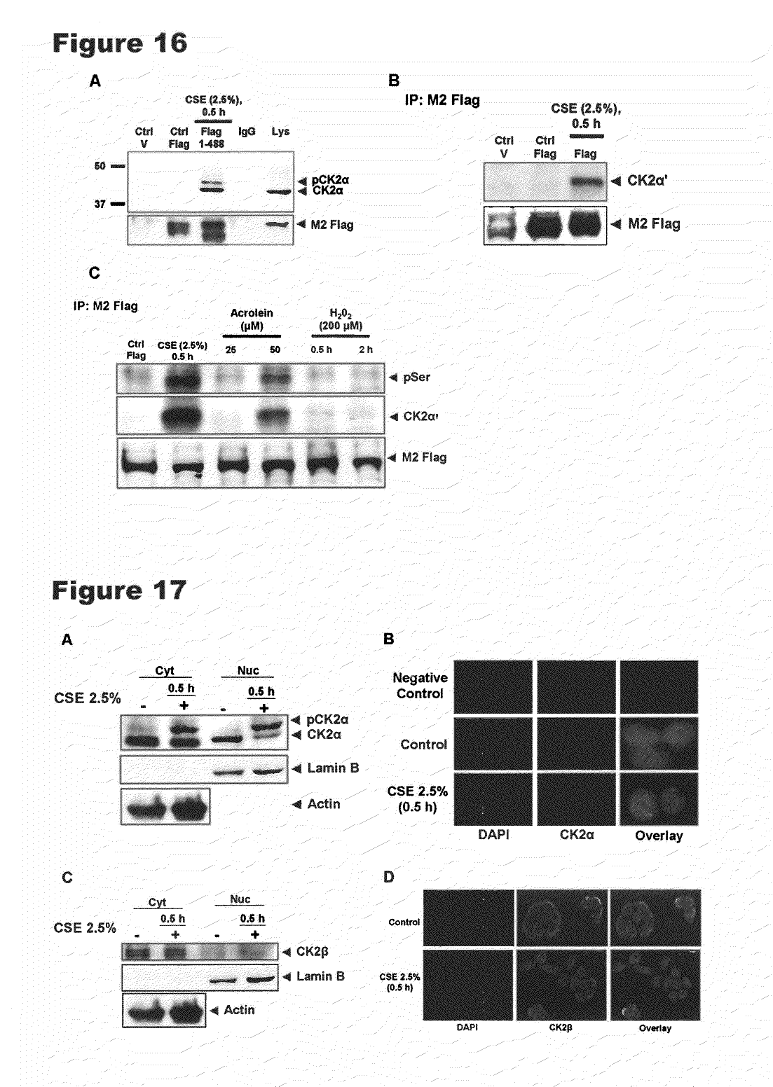

FIG. 16: HDAC2 serine phosphorylation is associated with increased interaction with catalytic subunits of protein kinase CK2 in response to CSE. A) Bronchial epithelial H292 cells transfected with either a control vector (control V) or flag-tagged WT HDAC2 were treated with 2.5% CSE for 0.5 h. Immunoprecipitated flag-tagged HDAC2 was then resolved on a 7.5% SDS/polyacrylamide gel and relative CK2α and B) CK2α′ expression was determined by western blotting. C) Bronchial epithelial H292 cells transfected with flag-tagged WT HDAC2 were treated with either 2.5% CSE for 0.5 h, 25 or 50 μM Acrolein for 1 h and 200 μM H₂0₂ for 0.5 and 2 h respectively. Immunoprecipitated flag-tagged HDAC2 was then resolved on a 7.5% SDS/polyacrylamide gel and relative CK2α′ expression was determined by western blotting.

FIG. 17: Required For CK2-Dependent HDAC2 Phosphorylation. A) 20 μg of cytoplasmic and nuclear extracts respectively from bronchial epithelial H292 cells treated with or without 2.5% CSE for 0.5 h, were resolved on a 7.5% SDS/polyacrylamide gel and relative CK2α expression was determined by western blotting. B) H292 cells cultured on 8-well chamber slides were treated with 2.5% CSE for 0.5 h, washed and fixed with 4% PFA. Slides were incubated with CK2α antibodies overnight at 4° C. Slides were mounted with antifade mounting solution and images analysed by an Olympus inverted microscope. C) 20 μg of cytoplasmic and nuclear extracts respectively from bronchial epithelial H292 cells treated with or without 2.5% CSE for 0.5 h, were resolved on a 7.5% SDS/polyacrylamide gel and relative CK2 μl expression was determined by western blotting. D) H292 cells cultured on 8-well chamber slides were treated with 2.5% CSE for 0.5 h, washed and fixed with 4% PFA. Slides were incubated with CK2β antibodies overnight at 4° C. Slides were mounted with antifade mounting solution and images analysed by a Leica confocal microscope.

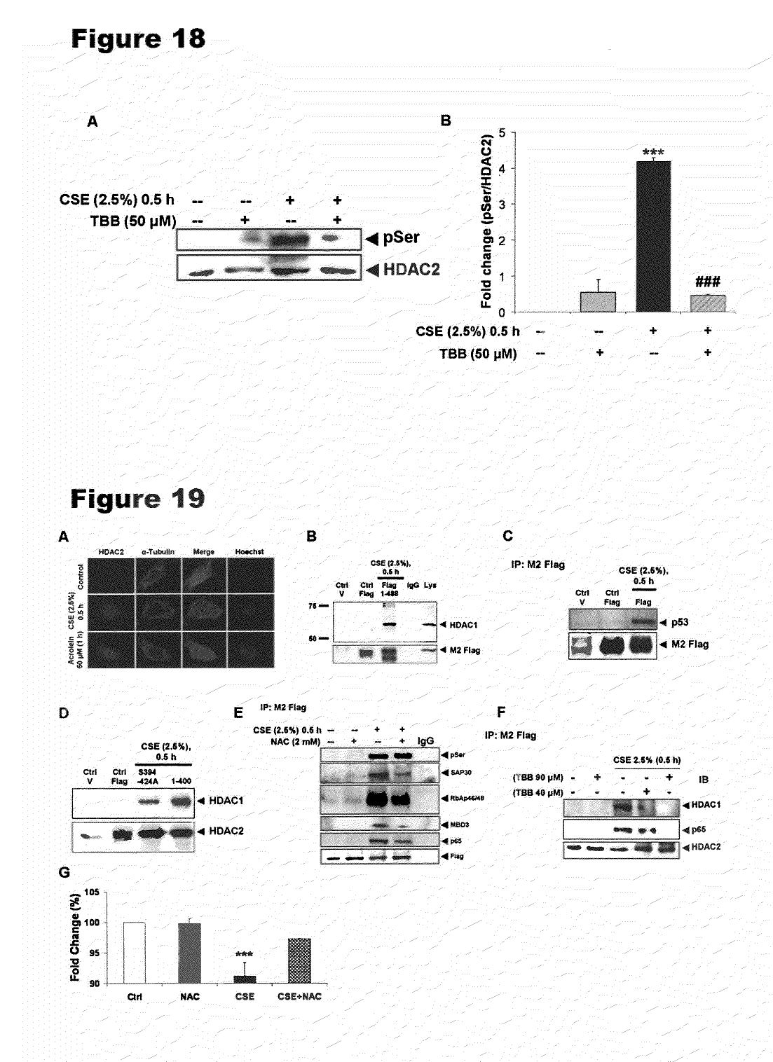

FIG. 18: Protein Kinase CK2 Inhibitors Blocks CSE-Induced HDAC2 Phosphorylation. A) Bronchial epithelial H292 cells transfected with a flag-tagged WT HDAC2 plasmid were treated with 2.5% CSE for 0.5 h. Immunoprecipitated flag-tagged HDAC2 was then resolved on a 7.5% SDS/polyacrylamide gel and relative phospho-serine expression was determined by western blotting using specific phospho-serine antibodies. B) Relative density of phospho-serine expression normalized to immunoprecipitated HDAC2. Data expressed as mean±SEM (n=3). *** P<0.001, ### P<0.001 significant compared to control treatments.

FIG. 19: CSE-Induced HDAC2 Phosphorylation is required for Co-Repressor Binding, Interaction with Transcription Factors but Does Not Affect Cellular Localization. A) H292 cells cultured on 8-well chamber slides were treated with 2.5% CSE for 0.5 h or 50 μM Acrolein for 1 h, washed and fixed with 4% PFA. Slides were incubated with HDAC2 and α-tubulin antibodies overnight at 40 C. Slides were mounted with antifade mounting solution and images analysed by an Olympus inverted microscope. B) H292 cells were transfected with plasmid vector (Ctrl V) or flag-tagged WT HDAC2 (1-488) for 24 h with lipofectamine 2000. Cells were treated with 2.5% CSE for 0.5 h and flag proteins immunoprecipitated using M2 anti-flag antibodies overnight at 4° C. HDAC1 and C) p53 interaction were determined by western blot. D) H292 cells were transfected with plasmid vector (Ctrl V), flag-tagged WT HDAC2 (1-488) or S³⁹⁴/⁴¹¹/⁴²²/⁴²⁴A and 1-400 amino acid HDAC2 mutants for 24 h with lipofectamine 2000. Cells were then exposed to 2.5% CSE for 0.5 h. Levels of HDAC2 interaction with HDAC1 were determined by western blotting using specific antibodies. E) H292 cells were transfected with flag-tagged WT HDAC2 (1-488) for 24 h with lipofectamine 2000. Cells were first pretreated with 2 mM NAC for 2 h, washed with 1×PBS and then exposed to 2.5% CSE for 0.5 h. Levels of phospho-serine and HDAC2 interaction with SAP30, RbAp46/48, MBD3 and p65 were determined by western blotting using specific antibodies respectively. F) H292 cells transfected with flag-tagged WT HDAC2 were pretreated with 40 μM and 90 μM 4,5,6,7-tetrabromobenzotriazole (TBB) for 2 and 0.5 h respectively. Cells were washed with 1×PBS and then exposed to 2.5% CSE for 0.5 h. Levels of HDAC1 and p65 binding to immunoprecipitated flag-tagged HDAC2 was determined by western blotting. G) H292 cells were pretreated with 2 mM NAC for 2 h, washed with PBS and then exposed to 2.5% CSE for 0.5 h. Deacetylase activity of immunoprecipitated HDAC2 was determined using a specific HDAC2 deacetylase activity kit. Data expressed as mean±SEM (n=3). *** P<0.001 significant compared to control treatments.

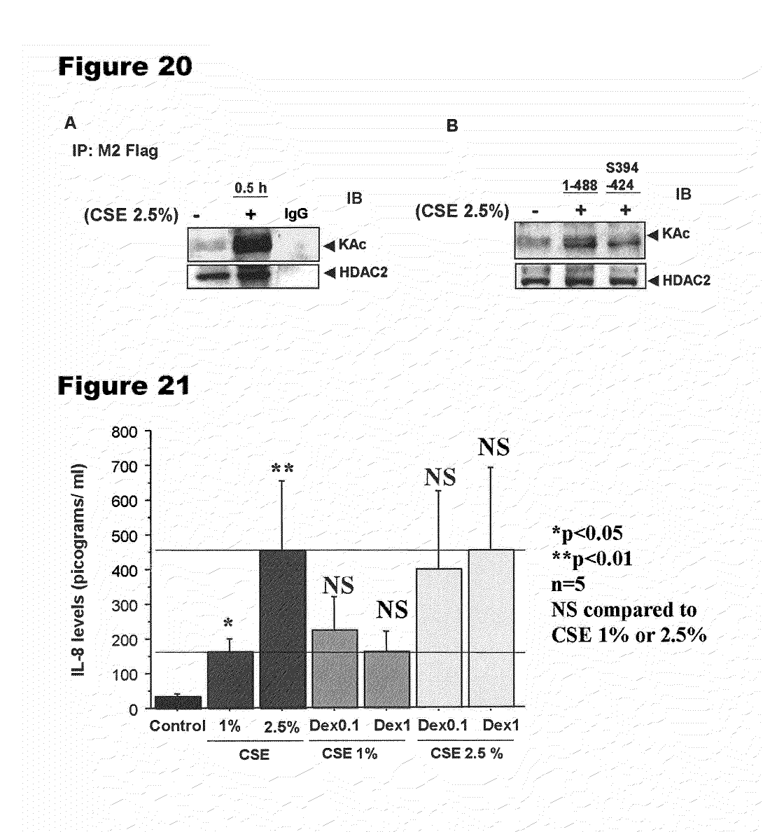

FIG. 20: HDAC2 is Acetylated in Response to CSE and this is Associated with Increased Phosphorylation. A) H292 cells were transfected flag-tagged WT HDAC2 (1-488) for 24 h with lipofectamine 2000. Cells were treated with 2.5% CSE for 0.5 h and flag proteins immunoprecipitated using M2 anti-flag antibodies overnight at 4° C. Lysine acetylation was determined by western blot using a monoclonal antibody. B) H292 cells were transfected with flag-tagged WT HDAC2 (1-488) or S³⁹⁴/⁴¹¹/⁴²²/⁴²⁴A HDAC2 mutants for 24 h with lipofectamine 2000. Cells were then exposed to 2.5% CSE for 0.5 h. Levels of lysine acetylation were determined by western blotting using specific antibodies.

FIG. 21: Dexamethasone Pretreatment does not Inhibit Cigarette Smoke Extract-mediated IL-8 release from MonoMac6 Cells at 24 Hours. Monocyte/Macrophage cell lines (MonoMac6) were pre-treated with either media alone or 0.1 and 1 dexamethasone for 2 h. Cells were then washed and treated with 1 and 2.5% CSE respectively for 24 h. Cell supernatants were assayed for IL-8 induction by sandwich ELISA using a commercially available kit. Data expressed as mean±SEM (n=3). * P<0.05, ** P<0.01, significant compared control treatments.

FIG. 22: HDAC2−/− mice are more susceptible to CS-induced lung inflammation. WT and HDAC2−/− mice were exposed to filtered air or acute CS for 3 days. Mice were sacrificed at 24 h post-last exposure, and lungs were lavaged. Bronchoalveolar lavage cells were prepared on cytospin slides and stained with Diff-Quik. (A) Total number of neutrophils in BALF. (B) MCP-1 levels were measured in mouse lung tissue by ELISA. Data are shown as mean±SEM (n=4 to 6). *P<0.05, **P<0.01, ***P<0.001 significant compared with corresponding air groups. #P<0.05, and ###P<0.001 compared with corresponding WT mice exposed to CS. $P<0.05, compared with air-exposed WT mice.

FIG. 23: HDAC2−/− mice are less responsive to budesonide in response to LPS-induced lung inflammation(A) WT mice were exposed to saline or aerosolized LPS (1 mg/ml). Nuclear extracts from mouse lungs were separated on SDS-PAGE gel, and HDAC2 protein levels were determined by immunoblotting. (B) Band intensity of HDAC2 levels normalized to actin. (C and D) WT and HDAC2−/− mice were pretreated with budesonide (3 mg/kg body weight) for 3 days at 1 h prior to aerosolization of LPS. (C) Neutrophils in BALF were determined following differential staining on cytospin slides. (D) MCP-1 levels were measured from lung homogenates of mouse lung tissue by ELISA. Data are shown as mean±SEM (n=4 to 6). ***P<0.001, significant compared with corresponding controls as denoted in figure. Sal: Saline; Bud: Budesonide.

FIG. 24: Increased lung inflammation is associated with HDAC2 reduction in Nrf2−/− mice exposed to CS (A) WT and Nrf2−/− mice were exposed to CS for 3 days. Differential counts from BALF were determined by Diff-Quik staining of cytospin slides. (B) Immunoprecipitated HDAC2 from lungs of naïve WT and Nrf2−/− mice were analyzed for deacetylase activity using specific HDAC deacetylase activity kit. (C) WT and Nrf2−/− mice were exposed to filtered air or whole body CS for 3 days. Lung tissue nuclear extracts were analyzed for HDAC2 relative expression. (D) HDAC2 levels normalized to actin expression. Data are shown as mean±SEM (n=4 to 9). *P<0.05, **P<0.01, and ***P<0.001, significant compared with corresponding air-exposed controls. #P<0.05 and ###P<0.001, significant compared with CS-exposed WT controls. $$$P<0.001, compared with air-exposed WT mice.

FIG. 25: Nrf2−/− mice are unresponsive to budesonide following LPS exposure. Nrf2−/− mice were treated with budesonide (1 or 3 mg/kg body weight) by intranasal administration followed by LPS exposure. (A) Percentage of neutrophils in BALF was determined following differential staining on cytospin slides. (B) MCP-1 levels were measured in lung tissue homogenates by ELISA. Data are shown as mean±SEM (n=4 to 6). ***P<0.001 significant compared with corresponding controls. Sal: Saline; Bud: Budesonide.

### DETAILED DESCRIPTION OF THE INVENTION

The present invention relates to methods for the regulation of the production of genes involved in inflammation by controlling the cellular levels or activity of histone deacetylase 2 (HDAC2). The methods of the present invention may be carried out by directly affecting HDAC2 or by affecting other upstream factors that subsequently lead to a change in the cellular levels or activity of HDAC2.

The term “cellular levels of HDAC2” as used herein may refer to the intracellular concentration of HDAC2. Cellular levels of HDAC2 may therefore be modified by causing and increase in production of the HDAC2 protein or by preventing the degradation of HDAC2 protein already present in the cell. The cellular level may also be increased functionally by causing more HDAC2 protein to be directed to a certain cellular compartment, such as the nucleus. In these cases, the total level of HDAC2 in the cell may not change, but the concentration of HDAC2 in the specific cellular compartment may be increased.

The term “activity of HDAC2” as used herein relates to the rate by which an HDAC2 protein catalyzes the deacetylation of histones. The activity of an HDAC2 proteins maybe increased though the contacting of the HDAC2 proteins with various compounds, such as co-factors and activators, which lead to an increased rate of catalysis for HDAC2.

Certain methods of the present invention are based, in part, on the discovery by the inventor that HDAC2 is phosphorylated and ubiquitinated upon exposure of the cigarette smoke, as is shown in the Examples below. Upon exposure to cigarette smoke, HDAC2 is phosphorylated by protein kinase CK2 (formerly known as casein kinase 2), which leads to subsequent ubiquitination and degradation of HDAC2. Without wishing to be bound by theory, the decreased levels of HDAC2 are thought to then lead to increased transcription of genes involved in inflammation through NF-κB driven promoters. The resulting inflammation leads to the development and progression of COPD and other diseases associated with inflammation, such as asthma, rheumatoid arthritis, and inflammatory bowel disease. The determination of this regulation of HDAC2 provides for several novel points of control of inflammation for the treatment of COPD and other diseases for which inflammation is controlled by HDAC2. Further description of the interaction of HDAC2, CK2, Nrf2 and other factors is given in the Examples below.

Certain other methods of the present invention are based, in part, on the discovery by the inventor that cellular HDAC2 levels are decreased in animals lacking Nrf2 and that animals lacking HDAC2 are more susceptible to cigarette smoke induced lung inflammation, as is shown in the Examples below. Without wishing to be bound by theory, the decreased levels of HDAC2 are thought to then lead to increased transcription of genes involved in inflammation through NF-κB driven promoters. The resulting inflammation leads to the development and progression of COPD and other diseases associated with inflammation, such as asthma, rheumatoid arthritis, and inflammatory bowel disease. The determination of this regulation of HDAC2 provides for several novel points of control of inflammation for the treatment of COPD and other diseases for which inflammation is controlled by HDAC2. Further description of the interaction of HDAC2, Nrf2 and other factors is given in the Examples below.

The methods of the present invention may be used for the treatment and/or prevention of COPD and other diseases. As the methods of the present invention reduce or eliminate the underlying cause of COPD, namely inflammation, the methods of the present invention can be preventative. If the methods of the present invention are begun while a subject is exposed to factors related to COPD development, such as cigarette smoke, but before the subject shows clinical signs of the disease (shortness of breath, wheezing, rapid breathing, coughing, etc.), the methods may be useful in preventing the clinical signs of the disease from ever manifesting or may slow the manifestation of the disease. The methods may also be used in treatment of COPD and other diseases once clinical signs have manifested. As the inflammation which is controlled by the present methods is necessary for the progression of COPD, the methods of the present invention may be used to slow or reverse the progression of the disease. The methods of the present invention may be able to reverse the progression of the disease to the point where the clinical symptoms are absent, whereby the subject will be considered cured or in remission.

In certain embodiments of the present invention, methods are provided whereby the cellular levels of HDAC2 are maintained through the use of CK2 inhibitors. As CK2 appears to be acting in its known role as a kinase in the regulation of HDAC2 levels, standard CK2 inhibitors known in the art may be used to treat or prevent COPD, either through the administration of one or more CK2 inhibitors alone or through the coadministration of one or more CK2 inhibitors along with a corticosteroid.

There are various CK2 inhibitors known in the art, including 1,3,8-trihydroxy-6-methyl-anthraquinone (emodin) and related compounds, such as aloe-emodin; flavinoids such as quercetin, fisetin and morin; and halogenated benzimidazole derivatives such as 4,5,6,7-tetrabromobenzotriazole (TBB), 4,5,6,7-tetrabromo-1H-benzimidazole (TBBz), 2-dimethylamino-4,5,6,7-tetrabromo-1H-benzimidazole (DMAT), and 5,6-dichloro-1-(β-D-ribofuranosyl)benzimidazole (DRB). Examples of CK2 inhibitors which may be used in the methods of the invention can be found in Sarno, et al., Pharmacology & Therapeutics, 2002:93:159-168, which is hereby incorporated by reference herein.

Also included among useful CK2 inhibitors are pharmaceutically acceptable molecular conjugates or salt forms of the inhibitors described above, that maintain activity as CK2 inhibitors as defined herein. Examples of pharmaceutically acceptable salts of CK2 inhibitors, include sulfate, chloride, carbonate, bicarbonate, nitrate, gluconate, fumarate, maleate, or succinate salts. Other embodiments of pharmaceutically acceptable salts contain cations, such as sodium, potassium, magnesium, calcium, ammonium, or the like. Other embodiments of useful CK2 inhibitors are hydrochloride salts. For providing enhanced cell permeability to a CK2 inhibitor moiety, various conjugated forms are useful, e.g., CK2 inhibitor-lipid conjugates, emulsified conjugates of CK2 inhibitors, lipophillic conjugates of CK2 inhibitors, and liposome- or micelle-conjugated CK2 inhibitors. (Fenske, D B et al., Biochim Biophys Acta, 2001:1512(2):259-72; Khopade, A J et al., Drug Deliv. 2000: 7(2):105-12; Lambert, D M et al., Eur. J. Pharm. Sci. 2000: 11 Suppl 2:S15-27; Pignatello, R et al., Eur J Pharm Sci. 2000: 10(3):237-45; Allen, C et al., Drug Deliv. 2000:7(3):139-45; Dass, C R et al., Drug Deliv. 2002: 9(1):11-8; Dass, C R, Drug Deliv. 2000:7(3):161-82; which are hereby incorporated by reference herein).

The CK2 inhibitors can be synthesized by known chemical means or can be procured commercially (e.g., Sigma-Aldrich). Emodin and some of its derivatives are also typically isolated from the root and rhizomes of Rheum palmatum (Polygonaceae) or from the leaves of Aloe vera, respectively, and can be purified by known means. (E.g., Mueller, S. et al., Drug Metabolism and Disposition 1998: 26(6): 540-46; Pecere, T et al., Cancer Res. 2000: 60:2800-04).

The CK2 inhibitors of he pharmaceutically acceptable composition contains the CK2 inhibitor and, optionally, contains pharmaceutically acceptable solvent(s), adjuvant(s) and/or pharmaceutically acceptable non-medicinal, non-toxic carrier(s), binder(s), thickener(s), and/or filler substance(s) that are known to the skilled artisan for the formulation of tablets, pellets, capsules, solutions, emulsions, suspensions, and any other form suitable for use. The carriers which can be used include glucose, lactose, sucrose, gum acacia, gelatin, mannitol, starch, starch paste, magnesium trisilicate, talc, corn starch, keratin, colloidal silica, potato starch, urea, medium chain length triglycerides, dextrans, petrolatum, and other carriers suitable for use in manufacturing preparations, in solid, semisolid, or liquid form. In addition auxiliary, stabilizing, thickening and coloring agents and perfumes can be used. Also contemplated are additional medicinal or nutritive additives in combination with at least one CK2 inhibitor, such as vitamins and the like.

The pharmaceutically acceptable composition containing the CK2 inhibitor may administered by any suitable method, including by oral, parenteral, inhalation and transdermal routes. Such methods of administering are well known to those of skill in the art.

The pharmaceutically acceptable compositions can be formulated for oral or enteral administration, for example, as tablets, troches, caplets, microspheres, hard or soft capsules, lozenges, aqueous or oily suspensions, dispersible powders or granules, emulsions, syrups, elixirs or enteral formulas.

Controlled release or continuous dosing regimens are also useful. The pharmaceutical industry has developed all sorts of slow and/or sustained-release technology. Sustained-release formulations employ several methods. The most common is a tablet containing an insoluble core; a drug applied to the outside layer is released soon after the medication is ingested, but drug trapped inside the core is released more slowly. Capsules containing multiparticulate units of drug with coatings that dissolve at different rates are designed to give a sustained-release effect.

The CK2 inhibitors of the present invention may also be formulated for administration by inhalation, as is well known in the art. The CK2 inhibitors may be formulated as are inhalable corticosteroids, such as in aerosol form, containing a propellant such as HFA-134a (1,1,1,2-tetrafluoroethane) or may be formulated with excipients such as lactose for inhalation. Examples of inhalable corticosteroids having suitable systems for administration by inhalation include AEROBID®, AEROSPAN®, FLOVENT®, FLOVENT ROTODISK®, PLUMICORT®, and PLUMICORT TURBUHALER®. The CK2 inhibitors of the present invention may be formulated with a corticosteroid, for co-administration, or, alternatively, the CK2 inhibitors may be formulated alone using an inhalation system similar to those used for inhalable corticosteroids. Further examples of inhalable formulations include those in which the active ingredient is entrapped in a liposome, such as those systems described in U.S. Pat. No. 5,192,528 to Kishore, which is hereby incorporated by reference herein.

In accordance with the invention, pharmaceutically acceptable compositions are formulated to deliver an effective dose of at least one CK2 inhibitor by the above-described or any other pharmaceutically acceptable systemic delivery system, preferably in an amount of about 10 to about 100 milligrams per kilogram of body mass per dose of CK2 inhibitor, more preferably about 20 to about 80 milligrams per kilogram of body mass per dose, and most preferably about 25 to about 50 milligrams per kilogram of body mass per dose. It is contemplated that one to two doses of the CK2 inhibitor may be delivered to the subject each day, although two to four or more daily doses are also in accordance with the present invention. The useful pharmaceutically acceptable composition can be formulated and manufactured at more than one concentration unit of CK2 inhibitor, such that modular incremental amounts of CK2 inhibitors are easily administered to subjects of various sizes as needed.

In accordance with the inventive method, administration by injection can be in a bolus or by infusion over a period of one to thirty minutes, and most preferably during a period of one to about fifteen minutes. If by infusion, the practitioner skilled in the art is also cautious in regulating the total infusion volume, rate of liquid infusion, and electrolyte balance to avoid adverse physiological effects related to these.

For example, for delivery by intravascular infusion or bolus injection into a subject, the CK2 inhibitor is preferably in a solution that is suitably balanced, osmotically (e.g., about 0.15 M saline) and with respect to pH, typically between pH 7.2 and 7.5; preferably the solution further comprises a buffer, such as a phosphate buffer (e.g., in a phosphate buffered saline solution). The solution is formulated to deliver a dose of about 10 to 100 milligrams of CK2 inhibitor per kilogram body mass in a pharmaceutically acceptable fluid volume over a maximum of about thirty minutes.

Further examples of dosage forms of CK2 inhibitors can be found in U.S. Published Patent Application 2004/0121968 to Ljubimov, et al., which is hereby incorporated by reference herein.

In other embodiments of the present invention, methods are provided for treating COPD and related diseases through the downregulation of CK2 expression. These methods, such as the use of antisense inhibition, prevent the expression of CK2, which in turn prevents the phosphorylation and degradation of HDAC2.

The present invention employs oligonucleotides for use in antisense inhibition of the function of RNA and DNA encoding CK2. In the context of this invention, the term “oligonucleotide” refers to an oligomer or polymer of ribonucleic acid or deoxyribonucleic acid. This term includes oligonucleotides composed of naturally-occurring nucleobases, sugars and covalent inter-sugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. A discussion of antisense oligonucleotides and some desirable modifications can be found in De Mesmaeker et al., Acc. Chem. Res. 1995: 28:366-374.

Specific examples of some preferred oligonucleotides envisioned for this invention include those containing modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH₂—N(CH₃)—O—CH₂ [known as a methylene (methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH₂. The amide backbones disclosed by De Mesmaeker et al. (Acc. Chem. Res. 1995: 28, 366-374) are also preferred. Also preferred are oligonucleotides having morpholino backbone structures (Summerton and Weller, U.S. Pat. No. 5,034,506). In other preferred embodiments, such as the peptide nucleic acid (PNA) backbone, the phosphodiester backbone of the oligonucleotide is replaced with a polyamide backbone, the nucleobases being bound directly or indirectly to the aza nitrogen atoms of the polyamide backbone (Nielsen et al., Science 1991, 254, 1497).

Oligonucleotides may also contain one or more substituted sugar moieties. Preferred oligonucleotides comprise one of the following at the 2′ position: OH, SH, SCH₃, F, OCN, OCH₃O(CH₂)ₙCH₃, O(CH₂)ₙNH₂ or O(CH₂)ₙCH₃ where n is from 1 to about 10; C₁ to C₁₀ lower alkyl, alkoxyalkoxy, substituted lower alkyl, alkaryl or aralkyl; Cl; Br; CN; CF3; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes T-methoxyethoxy [2′-O—CH₂ CH₂ OCH₃, also known as 2′-O-(2-methoxyethyl)] (Martin et al., Helv. Chim. Acta 1995:78:486). Other preferred modifications include 2′-methoxy (2′-O—CH₃), 2′-propoxy (2′-OCH₂ CH₂ CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-me pyrimidines, particularly 5-methyl cytosine (also referred to as 5-methyl-2′deoxycytosine and often referred to in the art as 5-me-C), 5-hydroxymethylcytosine (HMC), glycosyl HMC and gentobiosyl HMC, as well as synthetic nucleobases, e.g., 2-aminoadenine, 2-thiouracil, 2-thiothymine, 5-bromouracil, 5-hydroxymethyluracil, 8-azaguanine, 7-deazaguanine, N⁶ (6-aminohexyl)adenine and 2,6-diaminopurine. Kornberg, A., DNA Replication, W, H. Freeman & Co., San Francisco, 1980, pp 75-77; Gebeyehu, G., et al. Nucl. Acids Res. 1987, 15, 4513). A “universal” base known in the art, e.g., inosine, may be included. 5-me-C substitutions have been shown to increase nucleic acid duplex stability by 0.6°-1.2° C. (Sanghvi, Y. S., in Antisense Research and Applications, Crooke and Lebleu, eds., CRC Press, Boca Raton, 1993, pp. 276-278) and are presently preferred base substitutions.

Another modification of the oligonucleotides of the invention involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety (Letsinger et al. Proc. Natl. Acad. Sci. USA 1989, 86, 6553), cholic acid (Manoharan et al. Bioorg. Med. Chem. Let. 1994, 4, 1053), a thioether, e.g., hexyl-5-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al. Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantine acetic acid (Manoharan et al. Tetrahedron Lett. 1995, 36, 3651), a palmityl moiety (Mishra et al. Biochim. Biophys. Acta 1995, 1264, 229), or an octadecyl amine or hexylamino-carbonyl-oxycholesterol moiety (Crooke et al. J. Pharmacol. Exp. Ther. 1996, 277, 923). Oligonucleotides comprising lipophilic moieties, and methods for preparing such oligonucleotides are known in the art, for example, U.S. Pat. Nos. 5,138,045, 5,218,105 and 5,459,255.

The oligonucleotides of the invention may be provided as prodrugs, which comprise one or more moieties which are cleaved off, generally in the body, to yield an active oligonucleotide. One example of a prodrug approach is described by Imbach et al. in WO Publication 94/26764.

It is not necessary for all positions in a given oligonucleotide to be uniformly modified and, in fact, more than one of the aforementioned modifications may be incorporated in a single oligonucleotide or even within a single nucleoside within an oligonucleotide. The present invention also includes oligonucleotides which are chimeric oligonucleotides. “Chimeric” oligonucleotides or “chimeras,” in the context of this invention, are oligonucleotides which contain two or more chemically distinct regions, each made up of at least one nucleotide. These oligonucleotides contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Consequently, comparable results can often be obtained with shorter oligonucleotides when chimeric oligos are used, compared to phosphorothioate deoxyoligonucleotides hybridizing to the same target region. Cleavage of the RNA target can be routinely detected by gel electrophoresis and, if necessary, associated nucleic acid hybridization techniques known in the art. Typically, chimeric oligonucleotides are “gapped” oligonucleotides (or “gapmers”) in which a region of deoxynucleotides (the “gap”), preferably containing at least four contiguous deoxynucleotides, is flanked by regions of modified nucleotides, preferably 2′-sugar modified nucleotides. In a preferred embodiment, the flanking regions (or “wings”) contain 2′-alkoxy or 2′ alkoxyalkoxy modifications, more preferably 2′-methoxyethoxy. In preferred embodiments the backbone may be phosphorothioate throughout or may be phosphodiester in the “wings” and phosphorothioate in the “gap”. In other preferred embodiments, chimeric oligonucleotides may be “winged” oligonucleotides (or “wingmers” or hemichimeras) in which there is a deoxy “gap”, preferably at least 4 contiguous deoxynucleotides, flanked on either the 5′ or the 3′ side by a region of modified nucleotides. Again, the flanking region (or “wing”) preferably contains 2′-alkoxy or 2′ alkoxyalkoxy modifications, more preferably 2′-methoxyethoxy and the backbone may be phosphorothioate throughout or may be phosphodiester in the “wing” and phosphorothioate in the “gap”. Other configurations of chimeric oligonucleotide are also comprehended by this invention. These may involve other modifications of the sugar, base or backbone, preferably in the oligonucleotide wing(s).

The oligonucleotides in accordance with this invention preferably comprise from about 8 to about 30 nucleotides. It is more preferred that such oligonucleotides comprise from about 12 to 25 nucleotides. As will be appreciated, a nucleotide is a base-sugar combination suitably bound to an adjacent nucleotide through phosphodiester or other bonds.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; however, the actual synthesis of the oligonucleotides is well within the talents of those having routine skill in the field. It is also known to use similar techniques to prepare other oligonucleotides such as the phosphorothioates and alkylated derivatives.

In this embodiment of the invention, it is preferred to target specific genes for antisense attack. “Targeting” an oligonucleotide to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid from an infectious agent. In the present invention, the target is a nucleic acid encoding CK2. The targeting process also includes determination of a site or sites within this gene for the oligonucleotide interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Because, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding CK2, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. The open reading frame (ORF) or “coding region”, which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene) and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene). The transcription initiation site, or “5′ cap site” and the 5′ cap region (which encompasses from about 25 to about 50 contiguous nucleotides at the extreme 5′ terminus of a capped mRNA) may also be effective targets. mRNA splice sites may also be preferred target regions, and are particularly useful in situations where aberrant splicing is implicated in disease, or where an overproduction of a particular mRNA splice product is implicated in disease. Where gene deletion rearrangements exist, aberrant fusion junctions are also preferred targets. Once the target site has been identified, oligonucleotides are chosen which are sufficiently complementary to the target, i.e., hybridize sufficiently well and with sufficient specificity, to give the desired effect under conditions in which specific hybridization is desired, i.e., under physiological conditions in the case of in vivo assays or therapeutic treatment or, in the case of in vitro methods, under conditions in which the methods are used. In preferred embodiments of the present invention, the oligonucleotides are specifically hybridizable with a transcription initiation site, a translation initiation site, coding sequences and sequences in the 5′- and 3′-untranslated regions of mRNA encoding CK2.

As is described above, methods for determining an antisense oligonucleotide from the sequence of the gene encoding CK2 are known to those in the art. Specific examples of a CK2 antisense oligonucleotide SEQ ID NO: 1, are shown in Sayed, et al., Oncogene 2001:20:6994-7005, which is hereby incorporated by reference herein.

In the embodiments of the present invention whereby a CK2 antisense oligonucleotide is to be delivered to the cells, they may be delivered by formulating the oligonucleotide into a pharmaceutical formulation as is well known in the art. Oligonucleotides may be formulated into pharmaceutical formulations as is described above. A preferred pharmaceutical formulation containing a CK2 antisense oligonucleotide is that which targets delivery to the lungs, such as an inhalable formulation as is described above. Further descriptions of pharmaceutical formulations containing oligonucleotides may be found in the art, for example in U.S. Pat. No. 5,801,154 to Baracchini, et al., which is hereby incorporated by reference herein.

In preferred embodiments of the invention, CK2 antisense oligonucleotides are deliverable directly to the lungs through an inhalable system. Such systems are known in the art, and include the use of viral vectors and non-viral vectors such as lipid and polymer based vectors. These vectors may be used to deliver the antisense oligonucleotide or a DNA sequence encoding the antisense oligonucleotide directly to the lung tissues. Examples of viral and non-viral vectors for the delivery of antisense oligonucleotides to the lung are provided in Durcan, et al., Molecular Pharmaceutics 2008:5:559-566, which is hereby incorporated by reference herein. If viral vectors are used to deliver oligonucleotides, the vector typically contains a nucleotide sequence that is capable of expressing the olignucleotide after the vector has entered the cell, as is well known in the art.

In certain embodiments of the present invention, methods are provided whereby the cellular levels of HDAC2 are maintained through the use of Nrf2 activators. As Nrf2 appears to be acting in its known role as a nuclear factor in the regulation of HDAC2 levels, standard Nrf2 activators known in the art may be used to treat or prevent COPD, either through the administration of one or more Nrf2 activators alone or through the coadministration of one or more Nrf2 activators along with a corticosteroid.

There are various Nrf2 activators known in the art, including tert-butylhydroquinone (tBHQ), sulforaphane, Oltipraz (4-methyl-5-(2-pyrazinyl)-3-dithiolethione), bardoxolone methyl (also known as CDDO-Me or RTA 402) from Reata phaiinaceuticals, dihydro-CDDO-trifluoroethyl amide (dh404), resveratrol, anethole dithiolethione, 6-methylsulphinylhexyl isothiocyanate, curcumin, caffeic acid phenethyl ester, and 4′-bromoflavone.

Also included among useful Nrf2 activators are pharmaceutically acceptable molecular conjugates or salt forms of the activators described above, that maintain activity as Nrf2 activators as defined herein. Examples of pharmaceutically acceptable salts of Nrf2 activators, include sulfate, chloride, carbonate, bicarbonate, nitrate, gluconate, fumarate, maleate, or succinate salts. Other embodiments of pharmaceutically acceptable salts contain cations, such as sodium, potassium, magnesium, calcium, ammonium, or the like. Other embodiments of useful Nrf2 activators are hydrochloride salts. For providing enhanced cell permeability to a Nrf2 activator moiety, various conjugated fauns are useful, e.g., Nrf2 activator-lipid conjugates, emulsified conjugates of Nrf2 activators, lipophillic conjugates of Nrf2 activators, and liposome- or micelle-conjugated Nrf2 activators. (Fenske, D B et al., Biochim Biophys Acta, 2001:1512(2):259-72; Khopade, A J et al., Drug Deliv. 2000: 7(2): 105-12; Lambert, D M et al., Eur. J. Pharm. Sci. 2000: 11 Suppl 2:S15-27; Pignatello, R et al., Eur J Pharm Sci. 2000: 10(3):237-45; Allen, C et al., Drug Deliv. 2000: 7(3):139-45; Dass, C R et al., Drug Deliv. 2002: 9(1): 11-8; Dass, C R, Drug Deliv. 2000:7(3): 161-82; which are hereby incorporated by reference herein).

The Nrf2 activators can be synthesized by known chemical means or can be procured commercially.

The Nrf2 activators of he pharmaceutically acceptable composition contains the Nrf2 activator and, optionally, contains pharmaceutically acceptable solvent(s), adjuvant(s) and/or pharmaceutically acceptable non-medicinal, non-toxic carrier(s), binder(s), thickener(s), and/or filler substance(s) that are known to the skilled artisan for the formulation of tablets, pellets, capsules, solutions, emulsions, suspensions, and any other form suitable for use. The carriers which can be used include glucose, lactose, sucrose, gum acacia, gelatin, mannitol, starch, starch paste, magnesium trisilicate, talc, corn starch, keratin, colloidal silica, potato starch, urea, medium chain length triglycerides, dextrans, petrolatum, and other carriers suitable for use in manufacturing preparations, in solid, semisolid, or liquid form. In addition auxiliary, stabilizing, thickening and coloring agents and perfumes can be used. Also contemplated are additional medicinal or nutritive additives in combination with at least one Nrf2 activator, such as vitamins and the like.

The pharmaceutically acceptable composition containing the Nrf2 activator may administered by any suitable method, including by oral, parenteral, inhalation and transdermal routes. Such methods of administering are well known to those of skill in the art.

The pharmaceutically acceptable compositions can be formulated for oral or enteral administration, for example, as tablets, troches, caplets, microspheres, hard or soft capsules, lozenges, aqueous or oily suspensions, dispersible powders or granules, emulsions, syrups, elixirs or enteral formulas.

Controlled release or continuous dosing regimens are also useful. The pharmaceutical industry has developed all sorts of slow and/or sustained-release technology. Sustained-release formulations employ several methods. The most common is a tablet containing an insoluble core; a drug applied to the outside layer is released soon after the medication is ingested, but drug trapped inside the core is released more slowly. Capsules containing multiparticulate units of drug with coatings that dissolve at different rates are designed to give a sustained-release effect.

The Nrf2 activators of the present invention may also be formulated for administration by inhalation, as is well known in the art. The Nrf2 activators may be formulated as are inhalable corticosteroids, such as in aerosol form, containing a propellant such as HFA-134a (1,1,1,2-tetrafluoro ethane) or may be formulated with excipients such as lactose for inhalation. Examples of inhalable corticosteroids having suitable systems for administration by inhalation include AEROBID®, AEROSPAN®, FLOVENT®, FLOVENT ROTODISK®, PLUMICORT®, and PLUMICORT TURBUHALER®. The Nrf2 activators of the present invention may be formulated with a corticosteroid, for co-administration, or, alternatively, the Nrf2 activators may be formulated alone using an inhalation system similar to those used for inhalable corticosteroids. Further examples of inhalable formulations include those in which the active ingredient is entrapped in a liposome, such as those systems described in U.S. Pat. No. 5,192,528 to Kishore, which is hereby incorporated by reference herein.

In accordance with the invention, pharmaceutically acceptable compositions are formulated to deliver an effective dose of at least one Nrf2 activator by the above-described or any other pharmaceutically acceptable systemic delivery system, preferably in an amount of about 10 to about 100 milligrams per kilogram of body mass per dose of Nrf2 activator, more preferably about 20 to about 80 milligrams per kilogram of body mass per dose, and most preferably about 25 to about 50 milligrams per kilogram of body mass per dose. It is contemplated that one to two doses of the Nrf2 activator may be delivered to the subject each day, although two to four or more daily doses are also in accordance with the present invention. The useful pharmaceutically acceptable composition can be formulated and manufactured at more than one concentration unit of Nrf2 activator, such that modular incremental amounts of Nrf2 activators are easily administered to subjects of various sizes as needed.

In accordance with the inventive method, administration by injection can be in a bolus or by infusion over a period of one to thirty minutes, and most preferably during a period of one to about fifteen minutes. If by infusion, the practitioner skilled in the art is also cautious in regulating the total infusion volume, rate of liquid infusion, and electrolyte balance to avoid adverse physiological effects related to these.

For example, for delivery by intravascular infusion or bolus injection into a subject, the Nrf2 activator is preferably in a solution that is suitably balanced, osmotically (e.g., about 0.15 M saline) and with respect to pH, typically between pH 7.2 and 7.5; preferably the solution further comprises a buffer, such as a phosphate buffer (e.g., in a phosphate buffered saline solution). The solution is formulated to deliver a dose of about 10 to 100 milligrams of Nrf2 activator per kilogram body mass in a pharmaceutically acceptable fluid volume over a maximum of about thirty minutes.

In other embodiments of the present invention, methods are provided for treating COPD and related diseases through the upregulation of Nrf2 expression. In certain embodiments, it may be desired to upregulate Nrf2 expression in a the cells of a subject that poorly express Nrf2 or express Nrf2 in a less functional form.

In other embodiments of the invention, methods are provided whereby HDAC2 is provided to a subject in order to increase or maintain the level of HDAC2 in the cell. In further embodiments of the invention, methods are provided whereby Nrf2 is provided to a subject in order to increase of maintain the level of HDAC2 and Nrf2 in the cell. In still further embodiments of the invention, methods are provided whereby both HDAC2 and Nrf2 are provided to a subject in order to increase of maintain the level of HDAC2 and Nrf2 in the cell. Such methods can be implemented using standard gene therapy methods as are well known in the art. The methods known in the art can be viral or non-viral. Examples of gene therapy methods applicable to the methods of the present invention can be found in Phillips, Gene Therapy Methods, Methods in Enzymology vol. 346, Elsevier Science & Technology Books, 2002.

In preferred embodiments of the invention, the HDAC2 and/or Nrf2 genes are delivered directly to the lungs. Recently, methods have been developed in the art by which genes have been delivered directly to the lungs using aerosol-mediated gene delivery. These methods for gene delivery to the lungs should be adaptable for the delivery of HDAC2 and/or Nrf2 to the lungs. Examples of such methods can be found in Hwang, et al., Respiratory and Critical Care Medicine, 2009:179:1131-1140, and Vachani, et al., “Gene therapy for mesothelioma and lung cancer,” Am J Respir Cell Mol Biol. 2010 April; 42(4):385-93, which are hereby incorporated by reference herein.

The Nrf2 gene to be delivered in the methods of the present invention may by a nucleic acid encoding the full length amino acid sequence of Nrf2, as is shown in SEQ ID NO: 4, or may encode smaller functional version of Nrf2. It is also contemplated that the Nrf2 gene to be delivered may encode an amino acid sequence that is not 100% identical to SEQ ID NO:4, such as a sequence that is 70%, 75%, 80%, 85%, 90%, 95% or 97% or greater to SEQ ID NO:4.

The above methods describe embodiments of the invention, and it should be apparent to one of skill in the art that there are other methods, which are not expressly described herein, which fall within the scope of the invention. The Examples provided below are for illustrative purposes, and are not meant to limit the scope of the claims in any way.

In further embodiments of the invention, methods are provided whereby a ubiquitination inhibitor or a ubiquitin-proteosome inhibitor is provided to a subject in order to prevent the ubiquitination and degradation of HDAC2. This method will allow HDAC2 levels to be maintained in the lung cells.

The ubiquitination inhibitors of the present invention may be inhibitors which block ubiquitin ligase or ubiquitin conjugating enzymes. Other possible compounds include molecules that stimulate de-ubiquitinating enzymes, such as E2/E3 ubiquitin de-ligase known as cbl. More information on the ubiquitin system and the targeting of it with drug molecules can be found in Petroski, et al., BMC Biochemistry 2008: 9(Suppl I): S7, which is hereby incorporated by reference herein. Other examples of inhibitors include rhodadine derivatives as described in U.S. Published Patent Application US 2006/0276520 to Singh, et al., and small molecule compounds as described in U.S. Published Patent Application 2005/0009871 to Ramesh, et al., which are hereby incorporated by reference herein.

Ubiquitin-proteosome inhibitors may also be provided which prevent the degradation of the ubiquitinated proteins. Such inhibitors are well known in the art. In certain embodiments of the invention, the proteosome inhibitor is N-Acetyl-LeuLeu-NIe-CHO (ALLN). Other examples include leupeptin, calpain inhibitors I and II, MG115, MG132, PSI, peptide glyoxal, peptide α-ketomide, and peptide boronic esters. Other examples of inhibitors include boronic esters as described in U.S. Pat. No. 5,780,454 to Adams, et al., and the inhibitors described in Myung, et al., Medicinal Research and Reviews, 2001:21:245-273, which are hereby incorporated by reference herein.

The ubiquitination and ubiquitin-proteosome inhibitors of the present invention may be formulated into pharmaceutical formulations in a manner analogous to that described above for the formulation of CK2 inhibitors. In preferred embodiments, the ubiquitination and ubiquitin-proteosome inhibitors are formulated for delivery directly to the lungs, as is described above. However, other formulations of ubiquitination and ubiquitin-proteosome inhibitors are equally applicable to the methods of the present invention.

In other embodiments of the invention, methods are provided whereby HDAC2 is provided to a subject in order to increase or maintain the level of HDAC2 in the cell. Such methods can be implemented using standard gene therapy methods as are well known in the art. The methods known in the art can be viral or non-viral. Examples of gene therapy methods applicable to the methods of the present invention can be found in Phillips, Gene Therapy Methods, Methods in Enzymology vol. 346, Elsevier Science & Technology Books, 2002.

In preferred embodiments of the invention, the HDAC2 gene is delivered directly to the lungs. Recently, methods have been developed in the art by which genes have been delivered directly to the lungs using aerosol-mediated gene delivery. These methods for gene delivery to the lungs should be adaptable for the delivery of HDAC2 to the lungs. Examples of such methods can be found in Hwang, et al., Respiratory and Critical Care Medicine, 2009:179:1131-1140, which is hereby incorporated by reference herein.

The HDAC2 gene to be delivered in the methods of the present invention may by a nucleic acid encoding the full length amino acid sequence of HDAC2, as is shown in SEQ ID NO: 2, or may encode smaller functional version of HDAC2. It is also contemplated that the HDAC2 gene to be delivered may encode an amino acid sequence that is not 100% identical to SEQ ID NO:2, such as a sequence that is 70%, 75%, 80%, 85%, 90%, 95% or 97% or greater to SEQ ID NO:2.

In certain embodiments of the invention, the HDAC2 gene to be delivered encodes a version of the HDAC2 protein in which one or more CK2 phosphorylation sites are mutated. These phosphorylation sites include serine residues S³⁹⁴, S⁴²² and S⁴²⁴ of SEQ ID NO: 2. As is shown in the examples below, mutation of these residues to alanine prevents CK2 phosphorylation and subsequent degradation of HDAC2. It is contemplated that the HDAC2 gene to be delivered may encode a protein having one, two or three of the above serine residues mutated. The residues may be mutated to alanine, but may also be mutated to another residue that prevents phosphorylation at that site.

In further embodiments of the invention, methods are provided whereby activators of MAPK phosphatase 1 (MKP1, also known as DUSP1) are administered to a subject.

MKP1 can be activated using small molecule compounds which are capable of increasing the phosphatase activity of the enzyme, e.g., compounds that increase the rate of catalysis of dephosphorylation. Such activators include nitroalkenes and retinoids. These small molecule MKP1 activators may be delivered in pharmaceutical formulations in a manner analogous to the delivery of the CK2 inhibitors described above. As with the CK2 inhibitors, preferred delivery is directly to the lungs, such as by an inhalation route, but other forms of delivery are also contemplated.

It is also contemplated that MKP1 may be delivered to the subject through gene therapy methods. A description of such methods is provided above in the discussion of delivering HDAC2 genes to a subject. As with the delivery of HDAC2, it is preferred that a gene encoding MKP1 be delivered to the lungs using the methods described above.

The MKP1 gene to be delivered in the methods of the present invention may by a nucleic acid encoding the full length amino acid sequence of MKP1, as is shown in SEQ ID NO: 3, or may encode smaller functional version of MKP1. It is also contemplated that the MKP1 gene to be delivered may encode an amino acid sequence that is not 100% identical to SEQ ID NO:3, such as a sequence that is 70%, 75%, 80%, 85%, 90%, 95% or 97% or greater to SEQ ID NO:3.

It is also contemplated that alternate means of inducing MKP-1 activity may be used in the methods of the present invention, e.g. by inhibition of CK2 kinase and direct phosphorylation of MKP-1 by p42/p44 MAP kinase (ERK) pathway.

It is contemplated that the methods described above may be used in combination with one another in order to provide treatment of HDAC2 associated diseases, such as COPD. For the treatment of COPD, the methods of the present invention may be used in combination with known COPD treatments, such as corticosteroids and theopylline, or may be used without such treatment methods. Further, while the preferred subject for treatment with the methods of the present invention is a human, it is also contemplated that the methods may be used in the treatment of other mammals, particularly domestic animals such as horses, dogs and cats.

The above methods describe embodiments of the invention, and it should be apparent to one of skill in the art that there are other methods, which are not expressly described herein, which fall within the scope of the invention as is set forth in the claims below. The following Examples are for illustrative purposes, and are not meant to limit the scope of the claims in any way.

### EXAMPLES

### Example 1

Cigarette smoke (CS)-induced abnormal lung inflammation involves the reduction of histone deacetylase 2 (HDAC2) expression which is associated with steroid resistance in subjects with COPD and severe asthmatics who smoke cigarettes. However, the molecular mechanism of cigarette smoke-mediated reduction of HDAC2 is not clearly known. The inventor hypothesized that HDAC2 is phosphorylated and subsequently degraded by the proteasome in vitro in macrophages (MonoMac6), bronchial epithelial cells (H292) and primary small airway epithelial cells (SAEC) and in vivo in mouse lungs in response to CS exposure, Cigarette smoke extract (CSE) exposure in MonoMac6, H292 and small airway epithelial (SAEC) cells led to phosphorylation of HDAC2 on serine/threonine residues by a protein casein kinase 2 (CK2)-mediated mechanism, decreased HDAC2 activity and increased ubiquitin-proteasome-dependent HDAC2 degradation, protein casein kinase (CK2) and proteasome inhibitors significantly reversed CSE-mediated HDAC2 degradation whereas protein phosphatase inhibitor, okadaic acid, caused phosphorylation and subsequent ubiquitination of HDAC2. CS induced HDAC2 phosphorylation was more persistent in mouse lungs up to 4 months of chronic CS exposure, and mice showed significantly lower lung HDAC2 levels. Thus, CS-mediated downregulation of HDAC2 in vitro and in vivo involves the induction of serine/threonine phosphorylation and proteasomal degradation which may have implications for steroid resistance caused by cigarette smoke.

### Materials and Methods

Cell Culture

The human monocyte-macrophage cell line (MonoMac6) was established from the peripheral blood of a subject with monoblastic leukemia (21). Cells were maintained in RPMI 1640 medium (Life Technologies, Gaithersburg, Mo.) supplemented with 10% fetal bovine serum (HyClone Laboratories, Logan, Utah, 2 mM L-glutamine, 100 μg/ml penicillin, 100 U/ml streptomycin, 1% non-essential amino acids, 1% sodium pyruvate, 1 μg/ml human holo-transferrin, 1 mM oxaloacetic acid and 9 μg/ml bovine insulin. Cells were cultured at 37° C. in humidified atmosphere under 5% CO₂. Human bronchial epithelial cells (H292) were purchased from the American Type Tissue Culture Collection (Manassas, Va.), H292 cells were grown in RPMI-1640 supplemented with 10% fetal bovine serum (HyClone Laboratories), 2 mM L-glutamine, 100 μg/ml penicillin and 100 U/ml streptomycin in humidified atmosphere under 5% CO₂ at 37° C. Human primary small airway epithelial cells (SAEC) were derived from a single healthy non-smoker and purchased from Lonza (previously Cambrex, Walkersville, Md.) along with growth media (SAGM) bulletKit® supplemented with 52 μg bovine pituitary extract, 0.5 ng/ml human recombinant epidermal growth factor (EGF), 0.5 μg/ml epinephrine, 10 μg/ml transferrin, 5 μg/ml insulin, 0.1 ng/ml retinoic acid (RAJ, 6.5 ng/ml triidothyronine, 50 μg/ml gentamicin/amphotericin S (GA-1000) and 50 μg/ml fatty acid-free bovine serum albumin. Cells were cultured according to manufacturer's instructions for no more than seven passages.

Preparation of Aqueous CSE

Research-grade cigarettes (2R4F) with a filter from the Kentucky Tobacco Research and Development Centre at the University of Kentucky (Lexington, Ky.) were used to prepare CSE by slowly bubbling smoke from one cigarette into 10 ml of RPMI-1640 with 0.5% serum at a rate of 1 cigarette/min (OD at 320 nm: 0.74±0.02) according to the Federal Trade Commission protocol, each puff was of 2 s duration and 35 ml volume. The pH of cigarette smoke extract was adjusted to 7.4 and sterile-filtered through a 0.45 μm filter (25-mm acrodisc: Pall, Ann Arbor, Mich.) (11, 22). The extract, defined as 10% CSE, was used for all experiments within 15 minutes of preparation. Air was bubbled into 10 ml of RPMI-1640 with 0.5%, serum and pH adjusted to 7.4, filtered and used as a control medium.

Treatments

MonoMac6 cells were washed with Ca²⁺/Mg²⁺ free PBS and seeded at a density of 5.0×10⁶ cells in 100 mm culture plates in a total volume of 7 ml RPMI-1640 containing 0.5% FBS. Cells were treated with CSE (2.5%) or Okadaic acid (40 nM and 1 μM) (Alexis Biochemicals, San Diego, Calif.). For treatments with protein casein kinase CK2 inhibitor, 4,5,6,7-tetrabromobenzotriazole (TBB), cells were pretreated with 20 μM TBB (Sigma, St. Louis, Mo.) for 1 or 2 h, cells were then washed with sterile PBS before treatment with CSE. For treatments with proteasome inhibitor (N-Acetyl-Leu-Leu-Nie-CHO, ALLN, 1 μM or 20 μM) (Calbiochem, San Diego, Calif.), cells were pretreated 2 h followed by exposure to CSE. Primary human small airway epithelial cells (SAEC) were grown in 12-well plates containing SAGM (Lonza, previously Cambrex, Walkersville, Mo.) until confluency and then treated with CSE 0.1%, 0.5% and 1%) for 4 h. H292 cells were seeded at a density of 2.5×10⁶ cells in 60 mm culture plates in a total volume of 4 ml RPMI-1640 containing 0.5% FBS. Cells were treated with CSE (2.5%), washed in ice-cold sterile Ca²⁺/Mg²⁺ free PBS and lysed in RIPA buffer (50 mM Tris HCl pH 7.4, 150 mM NaCl, 0.25 mM EDTA, 5 mM NaF, 0.1% sodium deoxycholate, 1% Triton X-100); cell extracts and cell culture media were stored at −80° C. until analyzed.

Animal CS Exposure

Male and female A/J mice (Jackson Laboratories, Bar Harbor, Me.) at 8-10 weeks of age were exposed whole body to either filtered air (FA) or dilute, slightly aged mainstream CS from 1R3 non-filtered research grade cigarettes (27.1 mg total particulate matter yield per cigarette; University of Kentucky Tobacco Research and Development Centre (UKTRDC Lexington, Ky.) for 16 weeks as described (23). For 2, 4 and 10 week mouse CS exposures, 2R4F filtered research grade cigarettes (University of Kentucky Tobacco. Research and Development Centre [UKTRDC]) were used. Mice were allowed to acclimatize to CS exposure in the first week by exposing to 100 mg total particulate matter (TPM)/m³ followed by exposure to 250 mg TPM/m³ for 5 d/week, 6 h/day for using either cigarettes as described previously (23).

Lung Tissue Protein Extraction

Frozen lung tissue (100-150 mg) was homogenized in 0.5 ml of buffer A (10 mM HEPES (pH 7.8), 10 mM KCl, 2 mM MgCl₂, 1 mM dithiothreitol, 0.1 M EDTA, 0.2 mM NaF, 0.2 mM Na orthovandate, 1% (v/v) Nonidet P-40, 0.4 mM phenylmethyl sulfonyl fluoride, and 1 μg/ml leupeptin) on ice for 30 minutes. Homogenates were centrifuged at 2000 g in a bench-top centrifuge for 30 seconds at 4° C. to remove cellular debris. Supernatants were then transferred to a 1.7-ml ice-cold Eppendorf tube and centrifuged for 30 seconds at 13,000 g at 4° C.; supernatant was collected as a cytoplasmic extract. The pellet was resuspended in 50 μl of buffer C (50 mM HEPES (pH 7.8), 50 mM KCl, 300 mM NaCl 0.1 M EDTA, 1 mM dithiothreitol, 10% (v/v) glycerol, 0.2 mM NaF, 0.2 mM Na orthovanadate, and 0.6 mM phenylmethyl sulfonyl fluoride) and placed on the rotator in the cold room for 30 minutes, then centrifuged at 13,000 g in an Eppendorf tube for 5 minutes and the supernatant collected as the nuclear extract and kept frozen at −80° C. Whole cell lysate was extracted from lung tissue after homogenization in RIPA buffer (16,24).

Western Blotting

Cells were lysed in 100 μl RIPA buffer supplemented with protease inhibitor cocktail (leupeptin, aprotinin, pepstatin and PMSF) and stored at −80° C. Protein estimation was performed by the bicinchoninic (BCA) acid method as described by the manufacturer (Pierce, Rockford, Ill.), Whole cell lysates (10 μg-20 μg) were electrophoresed on 7.5% SDS-polyacrylamide gels, electro-blotted on PVDF membranes (Millipore, Burlington, Mass.), and blocked with 5% BSA in Tris-buffered saline (TBS) with 0.1% Tween-20 for 1 h. Membranes were then incubated with rabbit polyclonal HDAC2 (sc-7899), goat polyclonal HDAC1 (sc-6298) and HDAC3 (sc-8138) (Santa Cruz, Santa Cruz, Calif.) with a 1:1000 dilution in 5% BSA in TBS. After extensive washing, primary antibodies were detected with secondary antibodies linked to horseradish peroxidase (Dako, Carpinteria, Calif.) and bound complex detected with enhanced chemiluminescence (PerkinElmer, Waltham, Mass.).

Immunoprecipitation

Cell lysate (200 μg protein) was incubated for overnight with anti-HDAC2 (2 μg) at 4° C., then immunoprecipitated with Protein G-agarose beads for 1 h with constant agitation. Beads were washed at least four times with RIPA buffer, boiled for 10 minutes in 35 μl 2× sample buffer and resolved on a 7.5% SDS-polyacrylamide gel. Cell lysate controls were used. Blots were probed with p-serine (1:1000) and p-threonine (1:1000) (Cell Signaling, Boston, Mass.) monoclonal antibodies for studies on the post-translational modification of HDAC2.

ELISA

The levels of interleukin-8 (IL-8) in cell culture conditioned media, and KC, MCP-1 in mouse lung homogenates were assayed using an individual ELISA kit (R&D, Minneapolis, Minn.).

HDAC Activity Assay

HDAC2 was immunoprecipitated from cell lysates or lung homogenates (500 μg protein) as described above. Beads were washed and incubated with 150 μl of 1 mM COLOR DE LYS substrate (Biomol) for 80 minutes with rocking at 37° C. 30 μl aliquots from each sample were placed in 96-well plates and 20 μl HDAC specific buffer was added. 50 μA of COLOR DE LYS developer containing 2 mM trichostatin A (TSA) was then added and incubated for a further 20 minutes with rocking at 37° C. Color development was monitored at 405 nm. HDAC2 activity was expressed relative to standard curve generated from 0-500 μM COLOR DE LYS deacetylated standard.

Statistical Analysis

Data are expressed as mean±SE of triplicate experiments. Statistical analysis of significance was calculated using one-way Analysis of Variance (ANOVA) with STATVIEW software. IMAGE J software was used for densitometry analysis. * P<0.05, ** P<0.01, *** P<0.001.

### Results

Class 1 HDACs are Degraded in Response to Cigarette Smoke Extract (CSE) Treatment in MonoMac6 Cells

The effect of CSE-induced cellular regulation of class I histone deacetylases (HDACs) with specific emphasis on HDAC2 was first determined in macrophage cell lines (MonoMac6), Previously, the inventor had shown that CSE decreased HDAC2 levels at 4 h and 24 h in MonoMac6 cells (11). In this study, it was determined the time-dependent effect of CSE on class I HDAC levels MonoMac6 cells were exposed to CSE (2.5%) for 0.5, 1, 2, 4 and 6 h. CSE decreased HDAC1 and HDAC2 expression levels by almost 2-fold as early as 1 h post-CSE treatment while a significant 4-fold decrease in HDAC2 levels was observed at 6 h (FIG. 1A-B). Intriguingly, HDAC3 was even less sensitive to CSE treatment and was expressed in much lower levels as compared to HDAC1 and HDAC2. To determine whether class 1 HDAC degradation was due to decreased CSE-induced cell viability, the levels of lactate dehydrogenase (LDH) secreted into conditioned cell media was measured immediately after harvest. Cells treated with CSE (2.5%) at 6 h showed less than 10% cell death as compared to cells treated with media alone (data not shown).

To confirm that HDAC2 degradation was a direct consequence of CSE exposure, an optimal time point (6 h) was selected at which significant HDAC2 degradation was observed and MonoMac6 cells were exposed to increasing concentrations of CSE. Increasing doses of CSE exposure induced a dose-dependent decrease in HDAC2 levels (data not shown). MonoMac6 cells exposed to LPS (100 ng/ml) for 6 h were used as negative controls and no change in HDAC2 expression was observed compared to untreated cells.

Class 1 HDACs are predominantly nuclear proteins and do not possess a nuclear localization signal (8), but it is thought that class I HDACs are also present in the cytoplasm. To determine if CSE induced significant HDAC2 nucleo-cyloplasmic shuttling, MonoMac6 cells were treated with CSE (2.5%) for 4 and 6 h, cells were fixed in 4% paraformaldehyde and immunostained with anti-HDAC2 antibodies to determine HDAC2 localization. Contrary to expectations, HDAC2 did not show any significant translocation to the cytoplasm in response to CSE. To determine whether shuttling occurred at earlier time points subsequent to degradation of HDAC2, MonoMac6 cells were exposed to CSE (2.5%) for 0.5 h and 1 h (data not shown). CSE did not induce any significant translocation of HDAC2 to the cytoplasm and expression levels in the nucleus were dose-dependently decreased compared to control treatments (FIG. 1C).

CSE-Induced HDAC2 Degradation is Associated with Decreased Specific HDAC2 Activity in MonoMac6 Cells

To determine if CSE-induced loss of HDAC2 would be preceded by a change in specific HDAC2 activity, MonoMac6 cells were treated with CSE (2.5%) for 0.5, 1 and 2 h and endogenous HDAC2 was immunoprecipitated. HDAC2 activity was significantly decreased as early as 2 h post-CSE treatment (FIG. 2).

CSE Induces HDAC2 Phosphorylation in MonoMac6 Cells

In vitro kinase assays have shown that class I HDACs (18, 25-27) are phosphoproteins, but no study has been carried out to determine whether environmental stimuli, such as CS can trigger unknown pathways involved in HDAC2 phosphorylation and the physiological relevance of the regulation of HDAC2 via phosphorylation. It was hypothesized that HDAC2 would be directly phosphorylated in vivo by CS. To determine whether CSE phosphorylated HDAC2 in vivo, MonoMac6 cells were treated with CSE (2.5%) for 0.5, 1 and 2 h. CSE significantly induced the phosphorylation of immunoprecipitated HDAC2 on both serine and threonine residues (FIG. 3A-C). Since it had been initially hypothesized that HDAC2 phosphorylation would negatively regulate cellular HDAC2 levels and protein casein kinase CK2 had earlier been implicated both in in vitro and hypoxia-induced HDAC2 phosphorylation (18, 28), it was reasoned that CK2 inhibition would attenuate CSE-induced HDAC2 degradation, MonoMac6 cells were pre-treated for 2 h with a specific CK2 inhibitor, 4,5,6,7-tetrabromobenzotriazole (TBB, 20 11M), before treatment with CSE (2.5%) for 6 h. CK2 inhibition significantly attenuated HDAC2 degradation in response to CSE treatment (FIG. 3 D-E). To further validate the CSE induced phosphorylation of HDAC2 observed, MonoMac6 cells were treated with the PP1/2A phosphatase inhibitor, okadaic acid (1 μM), for 0.5 and 1 h. Immunoprecipitated HDAC2 was then assessed for phosphorylation of serine residues. Okadaic add significantly induced the phosphorylation of HDAC2 on serine residues as earlier observed with CSE [FIG. 3F (i-ii)]. CSE-induced HDAC2 phosphorylation was transient and was diminished after a longer than 2 h exposure to CSE. HDAC2 phosphorylation in response to okadaic acid was also lost beyond 1 h as phosphatase inhibition induced cell death at the dose used.

To further confirm this hypothesis, it was determined if exposure to okadaic acid would induce ubiquitination and degradation of HDAC2. MonoMac6 cells were treated with or without okadaic acid (40 nM), at a dose which was not toxic to the cells (determined by trypan blue cell viability assay and observing cellular morphology) for 24 h. Immunoprecipitated HDAC2 was then assayed for the presence of ubiquitinated residues using specific ubiquitin anti-sera by western blot. Since HDAC2 is basally regulated by the ubiquitin-proteasome pathway (20), it was expected to see some ubiquitination of HDAC2 in untreated cells, However, okadaic acid significantly induced the ubiquitination of HDAC2 [FIG. 3F (iii-iv)). To further confirm the hypothesis, MonoMac6 cells were treated with okadaic acid (1 μM) for 0.5 h, cells were fixed in 4% paraformaldehyde and immunostained with anti-HDAC2 antibodies to determine HDAC2 localization. Compared to untreated controls, okadaic acid significantly induced the loss of nuclear HDAC2 in MonoMac6 cells (FIG. 3G). As seen earlier with cells treated with CSE, no nucleo-cytoplasmic shuttling of HDAC2 was observed

HDAC2 Degradation is Ubiquitin-Proteasome Dependent

Since HDAC2 is known to undergo basal turnover via the proteasome (20), it was hypothesized that CSE-induced HDAC2 degradation is dependent on the proteasome; to test this a specific proteasome inhibitor was used, N-Acetyl-Leu-Leu-Nle-CHO (ALLN) MonoMac6 cells were pretreated with ALLN (1 μM and 5 μM) for 2 h before treatment with CSE (2.5%) for 6 h. ALLN dose-dependently inhibited HDAC2 degradation in response to CSE (FIG. 4A-B), ALLN (20 μM) did not induce cell death nor was HDAC2 degraded in cells treated with the compound alone (data not shown).

To determine if CSE-induced HDAC2 degradation was dependent on an ubiquitin mediated pathway, endogenous HDAC2 from MonoMac6 cells treated with CSE with or without ALLN (1 μM) for 6 h was precipitated and assayed for the presence of ubiquitinated residues. CSE treatment significantly induced the ubiquitination of HDAC2 which was further enhanced by pre-treatment with ALLN (FIG. 4C-D), an important mechanism required to target specific proteins for proteasomal degradation.

CSE Time-Dependently Caused Degradation of HDAC2 Associated with Increased Cytokine Release in Bronchial Epithelial Cells and Primary Small Airway Epithelial Cells

While macrophages are the primary inflammatory cells recruited to the lungs in response to CS exposure, the lung epithelium is the primary site of lung injury caused by noxious gases and inhaled oxidants in the lungs. Cytokines and chemokines released by epithelial cells drive the recruitment of inflammatory cells into the lung. It was therefore important to determine the effects of CSE exposure on HDAC2 regulation in epithelial cells. Bronchial epithelial cells (H292) were exposed to CSE (2.5%) for 1, 4, 12 and 24 h. While epithelial cells showed less sensitivity to CSE as compared to MonoMac6 cells, CSE-induced HDAC2 degradation was observed as early as 4 h post treatment [FIG. 5A (i-ii)]. To further confirm results observed in H292 cells, human primary small airway epithelial cells (SAEC) were exposed to CSE (0.5% and 1%) for 4 h. Lower doses of CSE were used this time since the primary cells had been shown to be more sensitive compared to immortalized epithelial cell lines. CSE dose-dependently decreased HDAC2 levels compared to control treatments [FIG. 5B (i-ii)]. To determine whether HDAC2 degradation correlated with increased release of inflammatory mediators in epithelial cells, conditioned cell media were used to measure levels of the neutrophil chemoattractant interleukin-8 (IL-8). CSE dose-dependently increased IL-8 release in H292 cells (FIG. 5C).

CSE Induces HDAC2 Phosphorylation in Human Bronchial Epithelial and Small Airway Epithelial Cells

It was then determined whether CSE-induced phosphorylation of HDAC2 was specific to macrophage cell lines alone. Bronchial epithelial cells (H292) were exposed to CSE (2.5%) for 0.5, 1, 2 and 4 h. CSE transiently induced HDAC2 phosphorylation with the most intense serine phosphorylation observed in H292 cells as early as 0.5-1 h [FIG. 6A (i-ii)]. As was observed in MonoMac6 cells, HDAC2 phosphorylation was transient but cyclical. HDAC2 phosphorylation was also observed slightly at 4 h even though phosphorylation had returned to baseline by 2 h post-CSE exposure. Similarly, it was determined whether HDAC2 was phosphorylated in vitro in SAECs by CSE. SAECs were exposed to CSE (0.1%, 0.5% and 1%) for 4 h and immunoprecipitated HDAC2 was probed for the presence of phosphorylated serine residues. CSE dose-dependently induced HDAC2 serine phosphorylation similar to H292 cells [FIG. 6B (i-ii)]. One striking difference was that higher doses of CSE induced phosphorylation at earlier time points compared to lower doses used; an indication that an unknown mechanism whereby HDAC2 is phosphorylated was being induced by CSE in a dose-dependent manner.

To determine if CSE-induced HDAC2 phosphorylation was dependent on protein casein kinase CK2, H292 cells were pretreated with a highly selective CK2 inhibitor (29), (TBB (20 μM), for 1 h, cells were then treated with or without CSE (25%,) for 0.5 h. The CK2 inhibitor completely reversed CSE-induced HDAC2 phosphorylation (FIG. 6C-D) implicating CK2 as the main kinase involved in HDAC2 phosphorylation as earlier observed by Seta and colleagues (18).

CS Exposure Induces HDAC2 Degradation and Decreased Activity in Mouse Lungs

To determine the role of CS in the regulation of mouse lung HDAC2 expression and activity, A/J mice were exposed to CS for 2, 4, 10 and 16 weeks. CS time-dependently decreased HDAC2 expression in mouse lungs (FIG. 7A-B) as compared to that of mouse exposed to filtered air alone. To determine whether CS decreased HDAC2 activity in mouse lungs, endogenous HDAC2 was immunoprecipitated from whole mouse lung homogenates and HDAC2 activity was measured using a COLOR DE LYS colorimetric assay kit. CS-exposed mice at 4 and 10 weeks significantly showed decreased HDAC2 activity as compared to air-exposed mice (FIG. 7C).

CS Induces HDAC2 Phosphorylation in Mouse Lungs

To determine whether HDAC2 was phosphorylated in mice exposed to short- and long-term CS, A/J mice were exposed to CS for 2, 4, 10 and 16 weeks and immunoprecipitated whole lung HDAC2 was analyzed for phosphorylation of serine/threonine residues. HDAC2 was significantly phosphorylated in the lungs of mice exposed to CS from 2 to 16 weeks as compared to that of mouse exposed to filtered air alone (FIG. 8). Basal HDAC2 phospho-serine and phospho-threonine phosphorylation in all air exposed mice from 2-16 weeks was very minimal as observed in MonoMac6, H292 bronchial epithelial cells and small airway epithelial cells. This indicates that phosphorylation is a tightly controlled physiological response to CS exposure. Intriguingly, HDAC2 serine/threonine phosphorylation patterns observed in mice lungs closely mirrored patterns of HDAC2 degradation. Similar to what was observed in SAECs, increased HDAC2 phosphorylation correlated with an increased degradation of HDAC2 in mouse lungs. This signified an important linkage between HDAC2 phosphorylation and subsequent loss of activity and degradation of HDAC2. Another interesting finding was that phosphorylation seemed to be more pronounced on serine residues compared to threonine residues. Furthermore, degradation of lung HDAC2 was associated with increased airspace enlargement in mice (23). Interestingly, HDAC2 levels were restored in lungs of mice allowed to recover in fresh filtered air for 4 months after a 5 month chronic CS exposure, suggesting that CS-induced loss of HDAC2 is reversible (data not shown).

CS Exposure Induces Increased Cytokine Release in Mouse Lungs

Soluble fractions from homogenized mouse lungs were probed for the presence of pro-inflammatory cytokines. CS exposed mice showed significantly higher levels of KC and MCP-1 as compared to air exposed mice (FIG. 9A-B). Interestingly KC and MCP-1 levels were significantly different in mice as early as 2 weeks post CS exposure at which significant HDAC2 serine phosphorylation and HDAC2 degradation was observed, an indication that phosphorylation of HDAC2 and its subsequent degradation played a key role in the induction of lung tissue cytokines.

### Discussion

Smokers and severe COPD subjects, known to be unresponsive to corticosteroid therapy, present with decreased HDAC2 levels/expression and activity in alveolar macrophages and lungs (14). CSE-induced HDAC2 degradation is also observed to cause increased global histone H4 acetylation and increased HAT activity in alveolar epithelial A549 cells and monocyte-macrophage MonoMac6 cells (11, 16, 30). Relevant data suggest that phosphorylation is associated with decreased HDAC2 activity (19). The inventor sought to test the hypothesis that CS-induced downregulation of HDAC2 involves phosphorylation of HDAC2 particularly on serine residues. The resultant data show that CSE caused degradation of HDAC1, HDAC2 and HDAC3 in MonoMac6 cells and also induced a decrease in specific HDAC2 activity. Interestingly, HDAC3 was less sensitive to CSE exposure compared to other class I HDACs studied and was expressed at lower levels, indicating that HDAC3 does not play as prominent a role in regulating the inflammatory response in macrophage cells exposed to CSE. HDAC3 is also regarded as distinct from HDAC1 and HDAC2 as it is not found in either Sin3 or NURD complexes (31).

Phosphorylation of proteins in eukaryotic cells on serine, threonine or tyrosine residues is an important mechanism by which several cellular processes are regulated Phosphorylation may act as a master switch to turn on or off certain kinases, activate enzymes, produce conformational changes in proteins or act as a trigger to induce proteasomal degradation of certain proteins such as IκB-α. HDAC2 was first discovered to be a phospho-protein using in vitro kinase assays that suggested that protein kinase CK2 was the major source of HDAC2 kinase (18). Seto and colleagues showed that HDAC2 was specifically phosphorylated on serine but not on threonine or tyrosine residues (18). Morita and colleagues also implicated protein kinase CK2 in hypoxia-induced phosphorylation of serine residues on HDAC2 in tumor cells by knocking down CK2 expression which abolished HDAC2 phosphorylation (28). Seta and colleagues, using in vitro techniques, determined three potential phosphorylation sites (Ser³⁹⁴, Ser⁴²², and Ser⁴²⁴) on HDAC2 that are present within the recognition sequences for protein kinase CK2 which has been largely shown to be responsible for HDAC2 phosphorylation (18). However, the physiological relevance of HDAC2 phosphorylation remains largely controversial (18, 19, 28, 32). Significant phosphorylation of HDAC2 was observed in MonoMac6 and mouse lungs both on serine and threonine residues, phosphorylation of threonine residues in bronchial epithelial cells (H292) and small airway epithelial cells (SAEC) was however negligible potentially indicating that while serine phosphorylation is global, phosphorylation of threonine residues may be cell- and tissue-specific, This is reinforced in part by a study that showed that serine residues, but not threonine or tyrosine residues, on HDAC2 were phosphorylated by CK2 (18), Surprisingly, immunoprecipitated HDAC2 from mouse lungs showed increased phosphorylation both on serine and threonine residues with increased period of exposure, The disparity may be due to differences in experimental conditions and cell lines utilized lung tissues represent a more physiologically relevant model to study the regulation of HDAC2 via phosphorylation compared to cell fines in response to CS exposure, CSE induced the degradation of HDAC2 expression in MonoMac6 cells as quickly as 1 h post exposure while HDAC2 degradation was observed 2 weeks after CS exposure in mouse lung tissue. Previously, the inventor had shown that HDAC2 was degraded in mouse lungs even after 3 days post-GS exposure (13) immunoprecipitated HDAC2 from lungs of CS-exposed mice showed increased levels of serine phosphorylation as early as 2 weeks post-CS exposure, serine phosphorylation increased with increasing degradation of HDAC2 in the lungs and suggested a strong linkage between phosphorylation and CS-induced downregulation of HDAC2, The highest level of phosphorylation was observed in 16 weeks CS-exposed groups that also showed the highest level of HDAC2 degradation which was associated with airspace enlargement in mice (23).

While the mechanism of CS-induced HDAC2 phosphorylation remains unknown, it is possible that CS directly induces several candidate kinases that phosphorylate HDAC2 or indirectly acts as a protein phosphatase inhibitor. This possibility was tested by treating MonoMac6 cells with the PP1/2A inhibitor, okadaic acid, Okadaic acid induced HDAC2 phosphorylation on serine residues and caused loss of HDAC2 in the nucleus as early as 0.5 h post-inhibitor exposure. These data were supported by the finding of Galasinski et al who showed that okadaic acid exposure induced phosphorylation of HDAC2 as observed by gel mobility retardation and disruption of the HDAC co-repressor complex (19). The fact that CS induces HDAC2 phosphorylation on serine/threonine residues may be a key clue pointing to a kinase-mediated effect.

Protein kinase CK2 is the only known kinase that directly phosphorylates HDAC2 on three known recognition sites (18). The hypothesis of whether CK2 inhibitors would attenuate HDAC2 phosphorylation was tested. A specific CK2 inhibitor, 4,5,6,7-tetrabromobenzotriazole (TBB) significantly reversed CSE-induced HDAC2 phosphorylation. In MonoMac6 cells, CK2 inhibition attenuated HDAC2 degradation in response to CSE implicating phosphorylation as a potentially important step towards CSE-induced HDAC2 degradation. While the data suggests protein kinase CK2 may be involved in CSE mediated phosphorylation of HDAC2, the contributions of other potential kinases (PI-3K and MAPK) and their involvement in HDAC2 phosphorylation in these cells cannot be ruled out.

While Ser³⁹⁴, Ser⁴²², and Ser⁴²⁴ are all present within CK2 recognition motifs, Ser⁴¹¹ does not and may be potentially phosphorylated by PKA and PKC (18). Oxidative stress (H₂O₂) has been shown to induce the activation of PI-3K which was associated with reduced HDAC activity in U937 monocytic cell line. The PI-3K/Akt inhibitor, LY-294002, restored oxidant-mediated reduced HDAC2 activity, but the effect of PI-3K/Akt inhibition on CSE-mediated phosphorylation of HDAC2 was however not studied (33). Furthermore, it remains to be seen whether PI-3K/Akt inhibitors can directly inhibit HDAC2 serine phosphorylation and restore steroid efficacy in vivo in response to CS in mouse lungs. Understanding the involvement of specific kinase inhibitors will lead to the possibility of developing small molecule kinase inhibitors which may represent a novel mechanism important in restoring steroid efficacy in subjects with COPD by restoring both HDAC2 activity and protein levels in the lungs.

Basal HDAC2 turnover and valproic acid (VPA)-induced HDAC2 degradation is mediated by the proteasome and requires the E2 ubiquitin conjugase, Ubc8. It was next determined if the same mechanism was involved in CSE-mediated HDAC2 degradation in MonoMac6 cells (20). The resultant data showed that HDAC2 degradation in response to CSE involved the proteasome as ALLN, a potent proteasomal inhibitor, attenuated CSE induced HDAC2 degradation. Interestingly, treatment of MonoMac6 cells with phosphatase inhibitor also induced increased phosphorylation, ubiquitination and degradation of HDAC2. Ubc8 expression was low in MonoMac6 cells and no significant induction of Ubc8 was observed in response to CSE, indicating that this pathway was not required for both okadaic acid and CSE-induced HDAC2 degradation. Immunocytochemistry did not show any nucleo-cytoplasmic shuttling, but it remains highly possible that HDAC2 phosphorylation is a trigger for the recruitment of potentially nuclear ubiquitinating proteins involved in targeting HDAC2 for eventual degradation. It is known that 14-3-3ε protein binds to phosphoserine residues of HDAC4 and HDAC5, leading to nucleo-cytoplasmic shuttling, but not to HDAC1 or HDAC2 (34). This may possibly explain the absence of nucleo-cytoplasmic shuttling in phosphorylated HDAC2 unlike HDAC4 and HDAC5.

Apart from kinase inhibitors, activation or restoration of inactive phosphatases such as the MAPK phosphatase-1 (MKP1) could prove a potential mechanism of restoring HDAC2 levels/activity. This hypothesis is further bolstered by the observation that the PP1/2A inhibitor, okadaic acid, induced a very similar response to CSE exposure in cells and CS exposure is known to decrease expression of lung tissue protein phosphatases such as MKP1 in vivo (35). The recent FDA approval of the clinically available proteasome inhibitor, velcade (bortezomib), raises the potential of the use of specific proteasome inhibitors to attenuate CS-induced HDAC2 degradation, as was observed with ALLN, with the long term goal of restoring steroid function in subjects with COPD and asthmatics who smoke.

In conclusion, the data herein clearly show that HDAC2 is negatively regulated by cigarette smoke via the phosphorylation-ubiquitin-proteasomal pathway both in vivo and in vitro. It is further shown that phosphorylation of HDAC2 plays a major role in regulating HDAC2 cellular expression both in vivo and in vitro. New findings are beginning to indicate that previously unknown mechanisms of action of HDAC2 enhancers may be as kinase inhibitors or via enhancing phosphatases, particularly MKP1.

### Example 2

### COPD Lung Inflammation, HDAC2 and Steroid Resistance

Chronic Obstructive Pulmonary Disease (COPD) is a slowly progressive condition characterized by airflow limitation, which is largely irreversible and is thought to result from an abnormal inflammatory response to inhaled particles or gases, commonly cigarette smoke. It is a disabling condition associated with progressive breathlessness. It is the fourth leading cause of chronic morbidity and mortality in the United States. COPD will account for over 6 million deaths per year in 2020 (3^{rd} major cause of death), and move from the sixth- to the third-leading cause of death worldwide. In America, COPD affects 9% of residents aged 60 years and above and it ranked fourth in the recent morbidity survey of the elderly population. It is estimated that approx 23.4 million people in the US have COPD and the health burden is $36.1 billions per year. There is as such no specific treatment option currently available to stop the progression of the disease and the current treatment is exclusively palliative. Thus, new therapeutic measures are urgently needed. The argument here is that these are unlikely to be developed without a better understanding of the patho-physiology of the disease at the cellular and molecular levels, based on understanding of the molecular mechanism(s) of cigarette smoke mediated inflammatory response. Thus, understanding the cellular signaling pathways involved in its pathogenesis could lead to the development of therapeutic strategies for this widespread debilitating disease.

Smoking is the main etiological factor, however, the enigma is that only 15-20% of smokers develop the disease. The reason why only a proportion of cigarette smokers are susceptible and develop COPD is unclear at present. This is thought to be due to an underlying abnormal inflammatory response to inhalation of cigarette smoke, resulting in susceptibility to smoke-induced injury in those who develop this disease. Even after stopping smoking, the subsequent course may depend on an individual's cellular ability to control inflammatory processes and will determine whether the outcome is containment or disease. It is proposed that abnormal inflammatory response in response to smoking is due to oxidative effects of cigarette smoke. However, very little is known about the molecular mechanisms whereby cigarette smoke-mediated oxidative stress triggers abnormal lung inflammation only in a susceptible population.

COPD is the sixth cause of death in the world affecting 4-6% of people over the age of 45 years. Moreover, this prevalence is expected to rise in the near future and the World Health Organization predicts that it will be the third major cause of death worldwide by the year 2020. It constitutes a major financial burden to society through direct and indirect health care costs and importantly has a major adverse impact upon quality-of-life. The effect of smoking patterns in the past will contribute to COPD morbidity and mortality in the foreseeable future. Smoking is the cause of COPD in over 90% of cases. While the main cause of COPD is smoking, only 15-20% of smokers develop clinically significant disease and show a rapid decline in forced expiratory volume in one second (FEV₁) and development of the disease. Unfortunately, current treatment for COPD is exclusively palliative. Thus, new therapeutic measures are urgently needed. The argument here is that these are unlikely to be developed without a better understanding of the patho-physiology of the disease at the cellular and molecular levels, based on understanding of the molecular mechanism of cigarette smoke mediated inflammatory response in vitro and in vivo in an animal exposure model and in lung specimens from patients whose disease is fully characterized, both functionally and pathologically.

Why Glucocorticoids have Poor Efficacy to Control Inflammatory Responses in COPD: Role of Chromatin Remodeling in Cigarette Smoke Mediated Inflammation

Corticosteroids are potent anti-inflammatory agents. However, they do not work significantly to inhibit the anti-inflammatory response in patients with COPD. It has been suggested that oxidative stress may have a role in the poor efficacy of corticosteroids in COPD. ROS and cigarette smoke-mediated inhibition of HDAC-2 levels is also supported by the observations that various proinflammatory mediators, such as intercellular adhesion molecule-1 (ICAM-1), IL-8, IL-6, TNF-α, IL-1β, monocyte chemoattractant protein-1 (MCP-1), matrix metalloproteinases (MMPs) and heat shock proteins, which are increased in BAL fluid of smokers, and are also induced by inhibition of histone deacetylases in various cells. It has been shown that glucocorticoid suppression of inflammatory genes requires recruitment of HDAC2 to the transcription activation complex by the glucocorticoid receptor. This results in deacetylation of histones and a decrease in inflammatory gene transcription. A reduced level of HDAC-2 was associated with increased pro-inflammatory response and reduced responsiveness to glucocorticoids in alveolar macrophages obtained from smokers.

### Cigarette Smoke and Oxidative Stress Triggers Alteration in Histone Acetylation and Deacetylation in Alveolar Epithelial Cells

The effect of cigarette smoke extract/condensate (CSC/CSE) and oxidative stress on histone acetylation:deacetylation and pro-inflammatory gene transcription in A549 epithelial cells has been investigated. Our results showed that CSE at different doses and hydrogen peroxide (100 μM) caused an increase in histone acetylation associated with increased histone acetyltransferase activity. It was also found that CSE and H₂O₂ inhibited histone deacetylase (HDAC) activity and HDAC2 protein levels. CSE-mediated decrease in HDAC2 level was associated with posttranslational modifications by 4-hydroxy-2-nonenal and nitric oxide radicals generated by cigarette smoke exposure. These data suggest that CSE unwinds chromatin and allows transcriptional machinery to be opened for secondary stimuli to cause transcription of pro-inflammatory genes.

Overall, our data suggest that CSE “relaxes or unwinds” chromatin and allows transcriptional machinery to be opened for secondary stimuli to cause transcription of pro-inflammatory genes. Furthermore, it was demonstrated the role of oxidative stress in histone acetylation:deacetylation leading to lung inflammation. CSE-induced acetylation (increased HAT activity) may be due to recruitment of CREB-binding protein (CBP) in the transcriptional complex. CSE inhibited HDAC protein possibly by protein modifications (nitration and oxidation). HDAC recruitment by glucocorticoids is required in the transcriptional activator complex to inhibit the pro-inflammatory gene transcription. Inhibition and/or post-translational modifications of HDAC proteins may render glucocorticoids ineffective.

HDAC2 is regulated by phosphorylation and dephosphorylation of serine residues (FIG. 10). CS may cause activation of CK2 leading to phosphorylation of HDAC2 leading to its degradation via ubiquitination. The molecular mechanism of histone acetylation was studied in resected human lungs obtained from smokers and patients with COPD. Decreased histone deacetylase (HDAC2) expression in mouse exposed to CS (FIG. 11), and in smokers and patients with COPD, was shown. The activation of NF-κB in peripheral lung sections of smokers and in patients with COPD was assessed. Immunohistochemical staining revealed that p65 subunit of NF-κB was not only predominantly localized but also deeply stained in the alveolar type II and airway epithelial cells and macrophages in smokers. These observations were more pronounced in lungs of patients with COPD. It was further demonstrated that the levels of histone deacetylase (HDAC2) were decreased in airway and alveolar epithelial cells as well as in macrophages of smokers with and without COPD (FIG. 12). HDAC2 reduction was associated with NF-κB activation in lungs of mouse exposed to CS (FIG. 13).

### SUMMARY AND CONCLUSIONS

Our data suggest that cigarette smoke “relaxes or unwinds” chromatin and allows transcriptional machinery to be opened for secondary stimuli to cause transcription of pro-inflammatory genes. It was shown that cigarette smoke augmented inflammatory cells when the cells were primed with NF-κB associated with reduction in HDAC2 levels. Furthermore, it was demonstrated that the role of oxidative stress in histone acetylation:deacetylation leading to inflammatory cytokine release both in vitro in alveolar epithelial cells and in vivo in rat lungs in response to tobacco smoke. Our human studies also revealed activation of NF-κB, altered histone acetylation (histone H3 and H4 acetylation): deacetylation (HDAC2 reduction) in lung tissue and lung sections of smokers and patients with COPD. Cigarette smoke induced acetylation (increased HAT activity) is due to recruitment of CREB-binding protein (CBP) in the transcriptional complex. Cigarette smoke inhibited HDAC2 protein by protein modifications (nitration and oxidation). HDAC2 recruitment by glucocorticoids is required in the transcriptional activator complex to inhibit the pro-inflammatory gene transcription. Inhibition and/or post-translational modifications of HDAC2 protein may render glucocorticoids ineffective. Our data also determined the underlying cause of abnormal and sustained pro-inflammatory response and to establish the trigger mechanisms that are activated to enhance the expression of pro-inflammatory genes. Thus, understanding of the molecular mechanism of histone acetylation by cigarette smoke would allow identification of novel antioxidant and/or anti-inflammatory targets for interventions to prevent or ameliorate COPD. The research described here has the potential to lead to new approaches that would block some of the untoward health consequences of tobacco smoke.

### Example 3

### Cigarette Smoke, HDAC2 Signaling and Phosphorylation by CK2α, and Role of HDAC2 in Steroid Resistance

### 1. Cigarette Smoke Extract (CSE)-Induced HDAC2 Phosphorylation by CK2α Requires Novel Serine Sites and is Phosphatase-Independent: Involvement of Ser Residues S³⁹⁴, S⁴²², S⁴²⁴

As earlier studies had shown that histone deacetylase 2 (HDAC2) could be phosphorylated in-vitro on serine phospho-acceptor sites by protein kinase (CK2), it was hypothesized that cigarette smoke extract (CSE) would induce either a kinase or phosphatase mediated phosphorylation of HDAC2 on serine sites. Flag-tagged full length HDAC2 transfected into human bronchial epithelial cell lines (H292) was significantly phosphorylated on serine residues both in response to CSE and acrolein, a small unsaturated aldehyde and component of CSE (FIGS. 14A and 14B). Phosphorylation was abolished when lysates where treated with lambda-phosphatase after CSE exposure (FIG. 14C). Site-specific serine to alanine mutations on sites S³⁹⁴, S⁴¹¹, S⁴²² and S⁴²⁴ completely abolished CSE-induced HDAC2 phosphorylation (FIG. 1D), suggesting these sites are critical for HDAC2 phosphorylation in-vivo. This is a novel finding in regulation of HDAC2.

PP2A activity was not affected by CSE treatment at time points where HDAC2 phosphorylation was observed (FIG. 14E) indicating that CSE-induced HDAC2 phosphorylation was solely dependent on a kinase-mediated mechanism.

### A) Protein Kinase CK2 Catalytic Subunit is Phosphorylated in Response to CSE

Since serine sites S³⁹⁴, S⁴²² and S⁴²⁴, shown to be required for CSE-induced HDAC2 phosphorylation, are conserved protein kinase CK2 phospho-acceptor sites on HDAC2, it was speculated that CK2 would be involved in HDAC2 phosphorylation in response to CSE. Contrary to published reports indicating that the CK2 catalytic subunit is constitutively active, a doublet band for CK2α in response to CSE was consistently observed (FIG. 15A) with no significant change in relative expression. To confirm if this doublet was due to CK2α phosphorylation, immunoprecipitated CK2α from bronchial epithelial (H292) cells was determined to be phosphorylated on serine residues in response to CSE (FIG. 15B). Importantly, phosphatase treatment of CK2α immunoprecipitates completely abolished the doublet band in response to CSE (FIG. 15C). Okadaic acid, a specific PP2A inhibitor which our lab had earlier shown to induce HDAC2 serine phosphorylation, also induced CK2α phosphorylation (FIG. 15D).

B) HDAC2 Serine Phosphorylation is Associated with Binding to CK2 Catalytic Subunits

The presence of conserved protein kinase CK2 sites that were highly phosphorylated on HDAC2 in response to CSE, led us to hypothesize a potential direct interaction between HDAC2 and protein CK2 catalytic subunits or the holoenzyme. Flag-tagged WT HDAC2 plasmids were transfected into H292 bronchial epithelial cells and then treated with CSE for 0.5 h. Both catalytic subunits of protein kinase CK2, CK2α, CK2α′ and the serine-phosphorylated form of CK2α were recruited to HDAC2 in response to CSE treatment (FIGS. 16A and 16B). To determine if this interaction was specific only to CSE, bronchial epithelial (H292) cells were also treated with either 25 and 50 μM acrolein for 1 h or 200 μM H₂O₂ for 0.5 and 2 h respectively. Acrolein-induced HDAC2 phosphorylation was also associated with increased HDAC2 CK2α′ binding (FIG. 16C). However, the inability of H₂O₂ phosphorylate HDAC2, unlike acrolein, may suggest a mechanism that is not limited solely to the generation of reactive oxygen species alone. Compared with CSE treatment, HDAC2 phosphorylation seemed to correlate with the level of CK2α binding. This finding further indicated a potential role for CK2α in HDAC2 phosphorylation. This is the first demonstration of CK2α regulation of HDAC2 in any system.

C) CK2β is not Required for CK2-Dependent HDAC2 Phosphorylation

The regulatory subunit, CK2β, of protein kinase CK2 plays two critical roles; to increase catalytic activity of the catalytic subunits and target them to a substrate protein as a holoenzyme, catalytic subunits of CK2 have however been shown to independently interact with substrate proteins. Since HDAC2 is a predominantly nuclear protein while CK2β is mostly localized to the cytoplasm, it was speculated that CK2β must be translocated to the nucleus in response to CSE if it is required to target CK2 catalytic subunits to HDAC2. Using both western blot and immunofluorescence techniques, no significant translocation of CK2α to the nucleus was observed (FIGS. 17A and B). Interestingly, the fact that the nuclear pool of CK2α was more highly phosphorylated compared to the cytoplasmic pool indicated nuclear CK2α was much more crucial for CSE-induced HDAC2 phosphorylation.

However CK2β, was not detected either in the nucleus or bound to HDAC2 both by western blotting and confocal microscopy in response to CSE (FIGS. 17C and D) suggesting that independent catalytic subunits of protein kinase CK2 but not the holoenzyme was sufficient for CSE and acrolein-induced HDAC2 phosphorylation and substrate specificity.

### D) Protein Kinase CK2 Inhibitors Blocks CSE-Induced HDAC2 Phosphorylation

CSE-induced interaction between HDAC2 and catalytic subunits of protein kinase CK2 and the fact that the three key sites implicated in HDAC2 phosphorylation both in-vitro and in-vivo are CK2 consensus sites suggested CK2 might be directly responsible in phosphorylating HDAC2 in response to CSE and/or acrolein. To validate this hypothesis, bronchial epithelial H292 cells were pre-treated with a specific CK2 inhibitor, 4,5,6,7-tetrabromobenzotriazole (TBB) for 2 h prior to exposure to CSE. 50 μM TBB was sufficient at completely inhibiting CSE-induced HDAC2 phosphorylation (FIG. 18A). Doses as low as 25 μM had also been observed to inhibit HDAC2 phosphorylation but not as efficiently. This data suggested a central role for CK2 as the primary kinase responsible for HDAC2 phosphorylation in-vivo.

E) CSE-Induced HDAC2 Phosphorylation is Required for Co-Repressor Binding Interaction with Transcription Factors but does not Affect Cellular Localization

Since CAMKII-mediated serine phosphorylation of class II HDACs (HDAC4 and HDAC5) have been shown to induce their nucleo-cytoplasmic translocation, it was speculated that HDAC2 phosphorylation may be induce HDAC2 translocation to the cytoplasm. However HDAC2 remained localized to the nucleus in response to either CSE or acrolein at doses and time points that induced serine phosphorylation (FIG. 19A). To determine further physiological roles for CSE-induced HDAC2 phosphorylation, flag-tagged WT HDAC2 or S^{394/422/424A} and c-terminal HDAC2 deletion (1-400) mutants were transfected into H292 cells exposed to CSE for 0.5 h. Site-specific serine to alanine mutations on sites S³⁹⁴, S⁴¹¹, S⁴²² and S⁴²⁴ decreased HDAC1-HDAC2 binding compared to the 1-400 mutant which still retained active serine site S³⁹⁴ (FIGS. 19B and D). Phosphorylated HDAC2 was also observed to interact with p53, a known deacetylation substrate in response to CSE (FIG. 19C) indicating a possible role for HDAC2 phosphorylation in regulating other pathways involved in cell cycle regulation. Inhibiting CSE-induced HDAC 2 phosphorylation by increasing intracellular GSH via the addition of 2 mM N-acetyl cysteine (NAC) blocked HDAC2 interaction with co-repressor proteins such as SAP30 specific to the Sin 3 complex, MBD3 specific to the NuRD complex and p65, a known deacetylase substrate for class I HDACs 1, 2 and 3 (FIG. 19E). To determine if inhibition of protein kinase CK2 would also block HDAC2 interaction with co-repressor proteins and transcription factors, H292 cells were pre-treated at different time points and doses with TBB prior to exposure to CSE. CK2 inhibition blocked HDAC2 interaction with both HDAC1 and p65 (FIG. 19F) further validating a functional role for HDAC2 phosphorylation.

Post-translational modifications such as tyrosine nitrosylation and protein-adduct formation have been shown to negatively regulate HDAC2 deacetylase activity. However this occurs at much longer time points of exposure to CSE. At 0.5 h, it was speculated that any effect to HDAC2 deacetylase activity would be dependent on its phosphorylation status alone. A significant decrease in HDAC2 deacetylase activity was observed which was rescued by pre-treatment with NAC (FIG. 19G). This provides the evidence that thiol antioxidants may have beneficial role in regulation of HDAC2 signaling and hence steroid resistance.

F) HDAC2 is Acetylated in Response to CSE and this is Associated with Increased Phosphorylation

Even though previous publications had reported HDAC1 acetylation on lysine residues with dexamethasone treatment in NIH 3T3 cells, there has been no reports on acetylation of HDAC2 despite the presence of at least 12 c-terminal conserved lysine residues. It was thus hypothesized that HDAC2 would be acetylated in response to CSE. Consistent with our hypothesis, significant lysine acetylation was observed on flag-tagged immunoprecipitated HDAC2 in response to CSE treatment (FIG. 20A). To determine if acetylation was dependent or independent of CSE-induced HDAC2 phosphorylation, the level of acetylation between WT HDAC2 and HDAC2 S^{394/411/422/424}, a mutant which we had shown to be less phosphorylated in response to CSE, was compared. Significantly reduced acetylation in the phosphorylation-mutant HDAC2 compared to the WT suggested that acetylation requires active phosphorylation of HDAC2. Presently, it is hypothesized that HDAC2 phosphorylation induces increased HDAC2 interaction with intrinsic histone acetyl transferases such as CBP/p300. The presence of p65 binding to HDAC2 in response to CSE further bolsters this possibility. Consistent with what was observed with HDAC1 acetylation, it is possible that acetylation might be directly responsible for the reduced HDAC2 deacetylase activity observed with increased phosphorylation. HDAC2 acetylation renders steroid inactive because acetylated HDAC2 will not be recruited into the corepressor complex.

G) Steroid-Dexamethasone Pretreatment does not Inhibit Cigarette Smoke Extract-Mediated IL-8 Release from MonoMac 6 Cells at 24 Hours: Novel Role of HDAC2 in Steroid Resistance

Loss of HDAC2 or post-translational modifications that negatively regulate HDAC2 deacetylase activity has long been implicated in steroid resistance. Since dexamethasone, a widely used corticosteroid used in anti-inflammatory therapy had been shown to significantly inhibit pro-inflammatory cytokine release in response to TNF-α and lipopolysacharide (LPS), it was hypothesized that a loss of HDAC2 would render dexamethasone ineffective at blocking IL-8 release in response to CSE. As expected, both low and high dose dexamethasone failed to respond to CSE treatment in the monocyte/macrophage cell line (Mono Mac 6) (FIG. 21) correlating with observed decreased relative expression of HDAC2. Since the inventor had earlier shown that inhibition of protein kinase CK2 significantly blocked CSE-induced HDAC2 degradation, it is possible that HDAC2 phosphorylation plays a significant role in inflammation-induced steroid resistance.

### Example 4

### Nrf2 Deficiency Influences Susceptibility to Steroid Resistance via HDAC2 Reduction

### Materials and Methods

Materials. Unless otherwise stated, all biochemical reagents used in this study were purchased from Sigma (St. Louis, Mo.). HDAC2 antibody was obtained from Santa Cruz Biotechnology (Santa Cruz, Calif.). Monoclonal β-actin antibody was obtained from Calbiochem (La Jolla, Calif.).

Mice. The Nrf2 knockout (KO) mouse strain (Nrf2−/− on C57BL/6J background) used in this study is described earlier [38] and was generously supplied by Prof. Masayuki Yamamoto, University of Tsukuba, Japan via the RIKEN BioResource Center, Tsukuba, Japan. HDAC2 mutant (HDAC2−/− on C57BL/6J background) mice were kindly provided by Dr. J. A. Epstein (University of Pennsylvania School of Medicine, Philadelphia, Pa.) [47]. These mice express a truncated and catalytically inactive form of HDAC2 with exons 9-14 replaced by a LacZ fusion gene created via a gene-trap method [47]. Wild-type (WT) C57BL/6J mice were purchased from Jackson laboratories (Bar Harbor, Me.). All animal protocols for this study were approved by the University Committee on Animal Research of the University of Rochester.

Cigarette smoke and lipopolysacharide (LPS) exposure to mice. Eight to ten week-old adult wild-type (WT), Nrf2−/− and HDAC2−/− were exposed to diluted mainstream CS generated from 3R4F filtered research grade cigarettes as described previously [11,13] for 1 h twice daily with 1 h interval in between for 3 days using a Baumgartner-Jaeger CSM2072i cigarette smoke-generating machine (CH Technologies, Westwood, N.J.). Smoke concentration and airflow were adjusted to obtain a constant particulate matter concentration of 300 mg/m3 total particulate matter (TPM) [11,13]. Mice exposed to filtered air served as controls. Mice were sacrificed at 24 h post-last CS exposure.

LPS exposure studies in mice were performed as described previously [48]. Briefly, age-matched WT, Nrf2−/− and HDAC2−/− were exposed to aerosolized Escherichia coli LPS (1 mg/ml) for 8 minutes. Aerosolized saline-exposed mice were used as controls, and animals were sacrificed at 24 h post last exposure.

Corticosteroid treatment. Budesonide in dry-powered form was dissolved in 70% ethanol and then diluted with saline prior to administration. Twenty-five microliters of budesonide solution, corresponding to 1 or 3 mg/kg body weight, was administered via an intranasal route to each mouse for 3 days followed by LPS exposure at 1 h after last budesonide treatment [49].

Bronchoalveolar lavage. Mice were anaesthetized by pentobarbital (Abbott Laboratories, Abbott Park, Ill.) intraperitoneal injection (100 mg/kg body weight) before sacrifice. Lungs were then removed and lavaged three times with 0.6 ml of 0.9% sodium chloride with cannula inserted into the trachea. Total lavage fluid for each mouse was combined and then centrifuged. Supernatants were frozen at −80° C. until required for further analysis and cell pellets resuspended in 1 ml of saline, and total number of cells determined using a haemocytometer. Differential counts (minimum 500 per slide) were determined using Diff-Quik (Dade Behring, Newark, Del.)-stained cytospin slides.

Lung tissue protein extraction. Cytoplasmic and nuclear proteins were extracted from frozen lung tissue samples as described [50]. Whole cell lysate was extracted from lung tissue after homogenization in RIPA buffer [11,13].

Immunoblotting. Protein estimation was performed by the bicinchoninic acid (BCA) method as described by the manufacturer (Pierce, Rockford, Ill.). Mouse lung nuclear extracts (10 μg-20 μg) were electrophoresed on 7.5% SDS-polyacrylamide gels, electro-blotted on PVDF membranes (Millipore, Burlington, Mass.). Membranes were incubated with primary antibody in a 1:1000 dilution in 5% BSA in TBS. After extensive washing, primary antibodies were detected with secondary antibodies linked to horse-radish peroxidase (Dako, Carpinteria, Calif.) and bound complex detected with enhanced chemiluminescence (PerkinElmer, Waltham, Mass.).

Cytokine Analysis. Monocyte chemotactic protein 1 (MCP-1) levels were measured from mouse lung soluble protein by enzyme-linked immunosorbent assay (ELISA) with the duo-antibody kit (R & D Systems). Results were normalized to protein concentration per sample.

HDAC2 activity assay. HDAC2 was immunoprecipitated from lung homogenates (500 μg protein) by incubating overnight with anti-HDAC2 antibody (2 μg). Beads were washed and incubated with Color de Lys substrate (Biomol) for 80 minutes with rocking at 37° C. 30 μL aliquots from each sample were placed in 96-well plates and HDAC specific buffer added. Color de Lys developer was then added and incubated for a further 20 minutes with rocking at 37° C. Color development was monitored at 405 nm. HDAC2 activity was expressed relative to standard curve generated from 0-500 μM Color de Lys deacetylated standard.

Statistical analysis. Data expressed as mean±SEM. Statistical significance was calculated using one-way Analysis of Variance (ANOVA) with STATVIEW software. NIH ImageJ software was used for densitometry analysis. P<0.05 as significant compared to relative controls.

### Results

HDAC2−/− Mice are More Susceptible to CS-Induced Lung Inflammation

CS exposure induced neutrophil influx in bronchoalveolar lavage fluid (BALF) of WT mice, which was significantly increased in HDAC2−/− mice (FIG. 22A). Consistent with this finding, air- and CS-exposed HDAC2−/− mice showed significant increase in MCP-1, a monocyte chemoattractant (FIG. 22B); and KC, a neutrophil chemoattractant (data not shown), release in the lungs as compared to corresponding air- and CS-exposed WT mice, suggesting the susceptibility of HDAC2 deficient mice to CS-induced lung inflammation. HDAC2−/− mice are less responsive to budesonide in inhibiting LPS-induced lung inflammation

LPS was used as a different lung inflammation-triggering agent rather than CS since HDAC2 is inactivated and degraded in response to CS exposure both in mouse lungs in vivo, and in epithelial cells and macrophages in vitro [10, 11, 50, 51], whereas LPS aerosolization in WT mice had no effect on HDAC2 protein levels in mouse lung (FIGS. 23A and B). Not surprisingly, neutrophil counts in BALF were significantly reduced in budesonide-pretreated LPS-exposed WT mice as compared to saline-pretreated LPS-exposed WT mice (FIG. 23C).

Budesonide pretreatment had no effect on neutrophil counts in BALF of HDAC2−/− mice (FIG. 23C). However, pretreatment of budesonide significantly lowered the MCP-1 (FIG. 23D) and KC (data not shown) release in WT mouse lung in response to LPS exposure. The levels of MCP-1 in lungs of HDAC2−/− mice were reduced by budesonide in response to LPS exposure (FIG. 23D). However, the efficacy of budesonide in decreasing MCP-1 release in HDAC2−/− mice (26.4%) was lower than that in WT mice (49.4%, P<0.01). Budesonide, however, had no appreciable effect on KC release in response to LPS exposure in HDAC2−/− mice (data not shown). Consistent with the MCP-1 and KC data, matrix metalloproteinase 9 (MMP9) activity was significantly elevated in HDAC2−/− mouse lungs compared to WT lungs with only a minimal effect of budesonide treatment observed (data not shown). Overall, these data suggest that HDAC2 deficiency leads to steroid insensitivity in terms of attenuating lung inflammatory response.

Enhanced lung inflammation is associated with HDAC2 reduction in Nrf2−/− mice exposed to CS

We determined whether mice deficient in the antioxidant transcription factor, Nrf2, would be more susceptible to CS-mediated oxidative stress to cause lung inflammation. Neutrophil influx, as a marker for increased vascular permeability and pulmonary inflammation, was increased in lungs of Nrf2−/− mice as compared to WT mice in response to 3 days CS exposure (FIG. 24A).

HDAC2 protein level and deacetylase activity are significantly decreased in response to CS-mediated oxidative stress in mouse lungs [50], therefore, we speculated that HDAC2 protein levels in lung may be altered in Nrf2−/− mice, which are susceptible to oxidative stress. Naïve Nrf2−/− mouse lung showed decreased HDAC2 activity compared to WT mouse lungs (FIG. 24B). To determine if this difference was due to accelerated loss of HDAC2 protein in lungs of Nrf2−/− mice, we compared HDAC2 protein abundance in nuclear extracts of Nrf2−/− and WT mice. Consistent with reduction in HDAC2 activity, the HDAC2 protein level was also significantly decreased in lung nuclear extracts of Nrf2−/− mice as compared to WT mice in the absence of CS exposure (FIGS. 24C and D). Three days of CS exposure further decreased the levels of HDAC2 protein in Nrf2−/− mice compared to WT mice (FIGS. 24C and D). These results suggest that the loss of HDAC2 in Nrf2 deficient mice is a crucial component of increased susceptibility to oxidative stress-induced inflammatory response in the lungs.

Nrf2−/− mice are not responsive to budesonide in inhibiting LPS-induced lung inflammation Decreased HDAC2 protein levels and deacetylase activity in naïve Nrf2−/− mice suggested that these mice may also exhibit resistance to steroid-mediated attenuation of lung inflammation. As shown in FIG. 23C, budesonide (3 mg/kg) pretreatment attenuated lung neutrophil influx following LPS exposure by ˜50% in WT mice. However, the neutrophils influx in BALF was not altered by pretreatment of budesonide (1 and 3 mg/kg) in Nrf2−/− mice in response to LPS exposure (FIG. 25A). Similarly, pretreatment of budesonide did not reduce LPS-induced release of MCP-1 (FIG. 4B) and KC (data not shown) release in Nrf2−/− mouse lung. These results suggest that the inability of steroids to inhibit lung inflammation possibly be due to HDAC2 reduction in lungs of Nrf2 deficient mice.

### Discussion

Although HDAC2 has been implicated in increased susceptibility to inflammation and steroid resistance in vitro [9,11], no such studies are available to determine the role of HDAC2 in vivo in lung in response to inhaled toxicants. In this study, we used mice expressing mutant HDAC2 [47,52,53] to study the steroid resistance in response to CS exposure. HDAC2−/− mice showed increased neutrophil recruitment in the lung in response to CS as compared to WT mice exposed to CS. Neutrophil recruitment was correlated strongly with the release of KC and MCP-1 in lung tissue suggesting the susceptibility of these mice to augmentation of CS-induced inflammation.

Since CS induces degradation of other HDACs (HDAC1 and HDAC3) that may be crucial for inflammation [11], it was important to determine a specific role for HDAC2 in steroid resistance utilizing an exposure model with no effect on HDAC levels or activity. Hence, we hypothesized that HDAC2−/− mice would exhibit a poor response to budesonide in response to LPS exposure. As expected, budesonide had no significant effect in reducing KC release in HDAC2−/− mice lungs in response to LPS. Furthermore, budesonide partially blocked MCP-1 release in the lungs of HDAC2−/− mice versus complete inhibition of MCP-1 in WT mice. Budesonide was also ineffective in reducing MMP9 activity in the mutant mice. Thus, these data suggest that basal HDAC2 deficiency leads to increased inflammatory response in the lung which is further augmented by LPS exposure. Furthermore, steroids have poor efficacy in HDAC2-ablated mice in response to pro-inflammatory challenge in the lung.

Increased susceptibility to oxidative stress (or deficiency of antioxidants) may exhibit phenotypes similar to HDAC2−/− mice in terms of steroid resistance. We used Nrf2−/− mice, which are susceptible to oxidative stress/cigarette smoke, to study the steroid resistance in controlling the lung inflammatory response. Disruption of Nrf2 enhanced susceptibility to acute CS-induced neutrophilic inflammation which is consistent with an earlier report [46]. Surprisingly, in air-exposed Nrf2−/− mice, neutrophil infiltration and RelA/p65 phosphorylation on ser²⁷⁶ and ser⁵³⁶ (unpublished data) were markedly enhanced in the lungs. It is possible that loss of Nrf2 leads to activation of NF-κB by increasing the pool of CREB-binding protein (CBP) available to interact with RelA/p65 [54,55], and thus causing induction of NF-B-dependent pro-inflammatory genes. Our data suggest that Nrf2 deficiency led to increased lung neutrophil influx and basal activation of NF-κB, which are augmented by CS exposure.

The finding that loss of Nrf2 induces an oxidative-stress phenotype in the lungs led us to hypothesize that increased oxidative stress status of the lung in Nrf2−/− mice would induce a reduction in HDAC2 activity and level. Lungs from naïve Nrf2−/− mice showed marked reduction in HDAC2 abundance and subsequently reduced HDAC2 deacetylase activity compared to naïve WT mice. Consistent with these data, exposure of Nrf2−/− mice to CS rapidly accelerated the rate of HDAC2 degradation suggesting the oxidant burden in lungs of Nrf2−/− is a key factor in loss of HDAC2 with or without exposure to environmental toxicants. The reduction in levels of HDAC2 in Nrf2−/− mice alternatively suggests that ARE-mediated Nrf2 activation may be required for HDAC2 transcription. However, the molecular regulation of HDAC2 expression or transcription factor(s) binding site is not clearly known [53]. It may also be possible that nuclear Nrf2 stabilizes the HDAC2 co-repressor complex on pro-inflammatory genes, whereas deficiency of Nrf2 leads to degradation of HDAC2 and activation of NF-κB-CBP binding on pro-inflammatory promoters [54,55]. Our preliminary data support this contention showing activation of CBP in lungs of Nrf2 null mice (unpublished data).

We next determined whether the increased inflammatory response and decreased HDAC2 protein levels in Nrf2−/− mice render them resistant to steroids in inhibiting the inflammatory response. WT and Nrf2−/− mice were exposed to aerosolized LPS following intranasal budesonide treatment. LPS induced severe neutrophilic inflammation in mouse lungs (unlike macrophage influx by CS) with no subsequent reduction in HDAC2 protein expression. Pre-treatment of WT mice with a high dose of budesonide significantly blocked LPS-induced lung neutrophilic influx which is consistent with budesonide-mediated inhibition of lung tissue KC levels. However, LPS-exposed Nrf2−/− mice failed to respond to both low and high dose budesonide treatments in blocking neutrophil influx, attenuating KC and MCP-1 release, and inhibiting RelA/p65 nuclear accumulation (unpublished data) in the lung. The observation of steroid resistance in Nrf2−/− and HDAC2−/− mice with the finding that HDAC2 protein levels are diminished in Nrf2−/− mice suggests a strong in vivo linkage between HDAC2 abundance and steroid sensitivity via oxidative stress.

In conclusion, loss of HDAC2 is a critical factor in inhaled toxicant-mediated lung inflammation especially in regulating the anti-inflammatory effects of glucocorticoids in mouse lungs. Oxidative stress-susceptible Nrf2−/− mice showed reduced HDAC2 levels and deacetylase activity in lungs, and are susceptible to CS- and LPS-induced inflammation. Interestingly, the loss of Nrf2 potentially leads to steroid resistance due to HDAC2 reduction. We speculate that the high oxidant status in the lung of Nrf2−/− mice leads to inactivation of endogenous HDAC2 through post-translational modifications, such as nitrosylation of tyrosine residues and carbonylation of cysteine, histidine and lysine residues. This may then block the ability of ligand-bound glucocorticoid receptors to recruit active HDAC2 to promoters of pro-inflammatory genes. Nevertheless, the rapid loss of HDAC2 and NF-κB activation in CS-exposed Nrf2−/− mice suggest steroid resistance is clearly a multifactorial cascade of events that perhaps start with oxidant/antioxidant imbalance and then precipitates at a much more rapid decline in HDAC2. This may have implications for devising better therapies for patients who are refractory/insensitive to steroid treatments, such as patients with COPD, asthma, rheumatoid arthritis, and inflammatory bowel disease [38].

### REFERENCES

1. Pryor W A, et al., Ann NY Acad Sci 1993; 686: 12-27.
2. Rahman I, et al., Eur Respir J 2006; 28:219-242.
3. Rahman I, et al., Am J Respir Crit Care Med 2002; 166:490-495.
4. Barnes P J, Am J Respir Crit Care Med 2000; 161:342-344.
5. Chikanza I C, et al., Rheumatology (Oxford) 2004; 43:1337-1345.
6 Farrell R J, et al. J Endocrinol 2003; 178:339-346.
7. Ito K, et al. Mol Cell Biol 2000; 20:6891-6903.
8. de Ruijter A J, et al., Biochem J 2003; 370:737-749.
9. Ito K, et al., Faseb J 2001; 15:1 110-11 12.
10. Marwick J A, et al., Am J Respir Cell Mol Biol 2004; 31:633-642.
11. Yang S R, et al., Am J Physiol Lung Cell Mol Physiol 2006; 291:L46-57.
12. Cosio B G, et al., Am J Respir Grit Care Med 2004: 170:141-147.
13. Yao H, et al., Am J Physiol Lung Cell Mol Physiol 2008; 294:L1174-1186.
14. Ito K. et al, N Engl J Med 2005; 352: 1967-1976.
15. Ito K, et al., Am J Respir Crit Gare Med 2002; 166:392-396.
16. Yang S R, et al., Am J Respir Cell Mol Biol 2008; 38:689-698.
17. Rajendrasozhan S, et al, Antioxid Redox Signal 2008; 10:799-811.
18. Tsai S C, et al. J Biol Chem 2002; 277:31826-31833.
19. Galasinski S C, et al, J Biol Chem 2002; 277; 19618-19626.
20. Kramer O H, et al., Embo J 2003; 22:3411-3420.
21. Ziegler-Heitbrock H W, et al., Int J Cancer 1988; 41:456-461.
22. Kode A, et al., Respir Res 2006; 7: 132-154.
23 March T H, et al., Toxicol Sci 2006; 92:545-559.
24. Yao H, et al., Am J Pathol 2008; 172: 122-1237.
25. Lee H, et al., Mol Cell Biol 2004; 24: 765-773.
26. Pflum M K, et al., J Biol Chem 2001; 276: 47733-47741.
27. Zhang X, et al., Genes Dev 2005; 19: 827-839.
28. Pluemsampant S, et al., Int J. Cancer 2008; 122: 333-341.
29. Sarno S, et al., FEBS Lett 2001; 496: 44-48.
30. Moodie F M, et al., Faseb J 2004; 18: 1897-1899.
31. Knoepfler P S, et al., Cell 1999; 99: 447-450.
32. Sun J M, et al., J Biol Chem 2007; 282: 33227-33236.
33. Faitla M, et al, J Respir Crit Care Med 2007; 176: A45.
34. Grozinger C M, et al., Proc Natl Acad Sci USA 2000; 97: 7835-7840.
35. Liu C, et al, J Nutr 2004; 134: 2705-2710.
36. V. M. Keatings, et al., Am J Respir Crit Care Med 155 (1997) 542-548.
37. M. Hew, et al., Am J Respir Crit Care Med 174 (2006) 134-141.
38. P. J. Barnes, et al., Lancet 373 (2009) 1905-1917.
39. K. Itoh, et al., Biochem Biophys Res Commun 236 (1997) 313-322.
40. A. K. Jaiswal, Free Radic Biol Med 36 (2004) 1199-1207.
41. D. Goven, et al., Thorax 63 (2008) 916-924.
42. D. Malhotra, et al., Am J Respir Crit Care Med 178 (2008) 592-604.
43. U. Garbin, et al., PLoS One 4 (2009) e8225-e8236.
44. T. Harju, et al., Am J Respir Crit Care Med 166 (2002) 754-759.
45. M. Tomaki, et al., Pulm Pharmacol Ther 20 (2007) 596-605.
46. T. Rangasamy, et al., J Clin Invest 114 (2004) 1248-1259.
47. C. M. Trivedi, et al., Nat Med 13 (2007) 324-331.
48. H. Yao, et al., Am J Respir Cell Mol Biol 39 (2008) 7-18.
49. D. S. Southam, et al., Eur Respir J 32 (2008) 970-978.
50. D. Adenuga, et al., Am J Respir Cell Mol Biol 40 (2009) 464-473.
51. D. Adenuga, et al., Arch Biochem Biophys 498 (2010) 62-73.
52. S. Zimmermann, et al., Cancer Res 67 (2007) 9047-9054.
53. O. H. Kramer, Trends Pharmacol Sci 30 (2009) 647-655.
54. G. H. Liu, et al., Biochim Biophys Acta 1783 (2008) 713-727.
55. Z. Sun, et al., Mol Cell Biol 29 (2009) 2658-2672.

What is claimed is:

1. A method for the treatment or prevention of a disease related to reduced cellular levels of histone deacetylase 2 (HDAC2) in a subject, comprising:
providing to the subject a molecular compound capable of maintaining cellular levels of HDAC2, wherein the molecular compound capable of maintaining cellular levels of HDAC2 is an inhibitor of protein kinase CK2, or a pharmaceutically acceptable salt or molecular conjugate thereof.

2. The method of claim 1, wherein the molecular compound capable of maintaining cellular levels of HDAC2 is provided in a pharmaceutical formulation.

3. The method of claim 2, wherein the pharmaceutical formulation is an inhaled pharmaceutical formulation.

4. The method of claim 1, wherein the inhibitor of protein kinase CK2 is selected from the group consisting of: emodin, aloe-emodin, quercetin, fisetin, morin, 4,5,6,7-tetrabromobenzotriazole (TBB), 4,5,6,7-tetrabromo-1H-benzimidazole (TBBz), 2-dimethylamino-4,5,6,7-tetrabromo-1H-benzimidazole (DMAT), and 5,6-dichloro-1-(β-D-ribofuranosyl)benzimidazole (DRB).

5. The method of claim 1, wherein the disease related to reduced cellular levels of HDAC2 is selected from the group consisting of: chronic obstructive pulmonary disorder, corticosteroid resistant chronic obstructive pulmonary disorder, asthma, rheumatoid arthritis and inflammatory bowel disease.

6. The method of claim 1, wherein the subject is also administered a corticosteroid.

7. A method for the treatment or prevention of a disease related to reduced cellular levels of histone deacetylase 2 (HDAC2) in a subject, comprising,
providing to the subject a oligonucleotide capable of interfering with the expression of protein kinase CK2.

8. The method of claim 16, wherein the oligonucleotide is formulated in a pharmaceutical formulation.

9. The method of claim 17, wherein the pharmaceutical formulation is an inhaled pharmaceutical formulation.

10. The method of claim 16, wherein the oligonucleotide is encoded in a nucleic acid which is part of a viral vector.

11. The method of claim 19, wherein the viral vector is targeted to the lungs.

12. The method of claim 16, wherein the oligonucleotide is a nucleic acid comprising SEQ ID NO: 1.

13. A method for the treatment or prevention of a disease related to reduced cellular levels of histone deacetylase 2 (HDAC2) in a subject, comprising, providing to the subject a molecular compound which acts as an activator of Nuclear factor (erythroid-derived 2)-like 2 (Nrf2).

14. The method of claim 13, wherein the molecular compound is provided in a pharmaceutical formulation.

15. The method of claim 14, wherein the pharmaceutical formulation is an inhaled pharmaceutical formulation,

16. The method of claim 13, wherein the molecular compound is selected from tert-butylhydroquinone (tBHQ), sulforaphane, Oltipraz (4-methyl-5-(2-pyrazinyl)-3-dithiolethione), bardoxolone methyl (also known as CDDO-Me or RTA 402) from Reata pharmaceuticals, dihydro-CDDO-trifluoroethyl amide (dh404), resveratrol, anethole dithiolethione, 6-methylsulphinylhexyl isothiocyanate, curcumin, caffeic acid phenethyl ester, and 4′-bromoflavone.

17. The method of claim 13, further comprising providing to the subject a corticosteroid.

18. The method of claim 13, wherein the disease related to reduced cellular levels of HDAC2 is selected from the group consisting of: chronic obstructive pulmonary disorder, corticosteroid resistant chronic obstructive pulmonary disorder, asthma, rheumatoid arthritis and inflammatory bowel disease.
